(19) United States
(12) Patent Application Publication
(10) Pub. No.: US 2026/0235546 A1
(43) Pub. Date: Aug. 13, 2026

(54) Methods and Systems for Interrogating Electrochemical Sensors

(71) Applicant: The Regents of the University of California, Oakland, CA (US)

(72) Inventors: Brian Roehrich, Goleta, CA (US); Kaylyn Leung, Santa Barbara, CA (US); Kevin Plaxco, Santa Barbara, CA (US); Lior Sepunaru, Goleta, CA (US); Julian Gerson, Santa Barbara, CA (US); Tod Kippin, Santa Barbara, CA (US); Ruben William Kolkman, Santa Barbara, CA (US); Nicole Emmons, Oakland, CA (US)

(73) Assignee: The Regents of the University of California, Oakland, CA (US)

(21) Appl. No.: 19/473,461

(22) PCT Filed: Apr. 8, 2024
(86) PCT No.: PCT/US2024/023635

§ 371 Date: Oct. 7, 2025

Related U.S. Application Data
(60) Provisional application No. 63/496,257, filed on Apr. 14, 2023.

(51) Int. Cl. G01N 27/327; G01N 33/487

(52) U.S. Cl. CPC G01N 27/3277; G01N 33/48707

(57) ABSTRACT

Systems and methods for using electrochemical sensors to determine the amount of analyte in samples are described. Many embodiments provide methods for interrogating electrochemical sensors for determination analyte amounts without the need for calibrating the sensors. The determination of the analyte amounts can take place at an improved time resolution.

### CROSS-REFERENCE TO RELATED APPLICATIONS

The current application claims the priority to U.S. Provisional Patent Application No. 63/496,257 entitled “Improved Methods of Interrogating an Electrochemical Sensor” filed Apr. 14, 2023. The disclosure of U.S. Provisional Patent Application No. 63/496,257 is hereby incorporated by reference in its entirety for all purposes.

### STATEMENT OF FEDERALLY SPONSORED RESEARCH

This invention was made with government support under EB022015 awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Apr. 8, 2024, is named 08510PCT.xml and is 3 kilobytes in size.

### FIELD OF THE INVENTION

The present invention relates generally to electrochemical sensors for determining the amount of an analyte in a sample. More particularly, the invention provides methods for interrogating electrochemical sensors allowing for the determination of an analyte amount without the need for a sensor calibration step, and at a high time resolution.

### BACKGROUND

Electrochemical sensors have found utility in a broad range of applications including in the detection of target analytes in the environment, manufacturing process streams, and biological fluids for example. Advantageously, these sensors are able to provide continuous quantitative output allowing for the ongoing monitoring of the material under analysis.

A number of electrochemical sensor types function on the basis of a change in electron transfer kinetics in response to the selective binding of a target analyte. In some sensors the electron transfer kinetics between the sensor's redox reporter and its electrode increase, and vice-versa, with the mean change in transfer rate being dependent on the concentration of the target.

Electrochemical aptamer-based (EAB) sensors are one type of electrochemical sensor. The aptamer in these is an oligonucleotide of defined base sequence known to selectively interact with a target analyte. In one version, the aptamer is coupled to the surface of the working electrode and a redox reporter is coupled to the free end of the aptamer. Binding of analyte to the aptamer causes a conformational change in the aptamer thereby causing the redox reporter to move more proximal to the electrode. That movement in turn causes an increase in the rate of electron transfer (kₑₜ) between the redox reporter and the electrode. This change in kₑₜ, informs on the target analyte concentration in real time and without the addition of exogenous reagents.

EAB sensors can determine the level of clinically relevant analytes in biological fluids both in vivo and in vitro. EAB sensors may routinely provide clinicians with important information on an individual to assist in diagnosing a new medical condition, managing an existing condition, and informing regarding prognosis.

Interrogation of an EAB sensor to detect target analyte requires the input of electrical energy, with current output by the working electrode being used to determine the amount of target analyte in solution. Square wave voltammetry (SWV) is often used for the in vivo detection of analyte given its sensitivity to changes in electron transfer rate and its ability to correct for the drift in current output often seen in, for example, in vivo sensor placements. To explain, the amount of an analyte reported by an EAB sensor tends to drift downward over time due to ongoing loss of functional aptamer that is in contact with a bodily fluid. This signal drift is a problem where the amount of analyte is being measured continuously, such as for in vivo monitoring applications. Measuring sequential square wave voltammograms at two different frequencies, however, enables drift correction in an approach called kinetic differential measurements (KDM). KDM utilises the difference between SWV measurements taken at two frequencies to subtractively remove drift.

A problem with SWV arises in that two square wave voltammograms are required for each measurement point which in turn reduces the time resolution of such measurements. For many in vivo examples, the time resolution of this approach is between 6 and 22 seconds. This low time resolution may be detrimental in applications for which more continuous real-time date is necessary, such as in the monitoring of rapid physiological processes. Neurotransmitter release is one example of a physiological process that is much more rapid than this timescale.

A further problem is that SWV-based interrogation is highly sensitive to sensor-to-sensor fabrication variation arising from differences in the number of recognition elements on each working electrode. Because of this, sensors employing SWV must be individually calibrated before use to correlate a peak current with an analyte amount.

It is an aspect of the present invention to provide an improvement to prior art electrochemical sensor interrogation methods. It is a further aspect of the present invention to provide a useful alternative to prior art electrochemical sensor interrogation methods.

The discussion of documents, acts, materials, devices, articles and the like, is included in this specification solely for the purpose of providing a context for the present invention. It is not suggested or represented that any or all of these matters formed part of the prior art base or were common general knowledge in the field relevant to the present invention as it existed before the priority date of each provisional claim of this application.

### SUMMARY OF THE INVENTION

Some embodiments include a method for determining an amount of an analyte in a sample comprising:
interrogating a working electrode of an electrochemical sensor by applying a voltage perturbation thereto, the voltage perturbation being a sum of two or more sinusoidal waveforms, each of the two or more sinusoidal waveforms being of a different frequency; measuring voltage and/or current values across the working electrode and a counter electrode at each of the different frequencies simultaneously;
applying an integral transform method to the measured voltage and/or current values to generate an impedance spectrum by determining impedance at the frequencies of the two or more sinusoidal waveforms; and
using the impedance spectrum to determine the amount of analyte in the sample.

In some embodiments, the integral transform method is selected from a Fourier transform method, a fast Fourier transform method, a Laplace transform method, a Mellin transform method, a Hartley transform method, and a Chirplet transform method.

In some embodiments, the two or more sinusoidal waveforms are 3, 4, 5, 6, 7, 8, 9, 10, 11, 12, 13, 14, 15, 16, 17, 18, 19, 20 or more sinusoidal waveforms.

In some embodiments, the frequencies of the two or more sinusoidal waveforms define a frequency range that includes frequencies informative of electron transfer kinetics between a redox reporter of the electrochemical sensor and a surface of the working electrode.

In some embodiments, the frequencies of the two or more sinusoidal waveforms define a frequency range that excludes frequencies not informative of electron transfer kinetics between a redox reporter of the electrochemical sensor and a surface of the working electrode.

In some embodiments, the frequencies of the two or more sinusoidal waveforms define a frequency range that excludes frequencies that result in a drift error in the determined analyte concentration.

In some embodiments, the frequencies of the two or more sinusoidal waveforms are each less than 2000 Hz, 1900 Hz, 1800 Hz, 1700 Hz, 1600 Hz, 1500 Hz, 1400 Hz, 1300 Hz, 1200 Hz, 1100 Hz, 1000 Hz, 900 Hz, 800 Hz, 700 Hz, 600 Hz, 500, 400 Hz, 300 Hz, 200 Hz, or 100 Hz.

In some embodiments, the frequencies of the two or more sinusoidal waveforms are each between 1 Hz and 2000 Hz, or are each between 1 Hz and 1000 Hz, or are each between 10 Hz and 100 Hz.

In some embodiments, the frequencies of the two or more sinusoidal waveforms include a first frequency informative of electron transfer kinetics between a redox reporter of the electrochemical sensor and a surface of the working electrode, a second frequency that is higher than the first frequency, and a third frequency that is lower than the first frequency.

In some embodiments, the frequencies of the two or more sinusoidal waveforms include a lower frequency and one or more frequencies higher than the lower frequency, and each of the one or more frequencies higher than the lower frequency is a multiple of the lower frequency.

In some embodiments, the lower frequency is the lowest of the frequencies of the two or more sinusoidal waveforms.

In some embodiments, the step of using the impedance spectrum to determine the amount of analyte comprises comparing an impedance spectrum or part thereof resulting from a test sample, with an impedance spectrum or part thereof resulting from a control sample containing no analyte.

In some embodiments, the impedance spectrum resulting from a control sample and the impedance spectrum resulting from a test sample are both arranged as frequency versus phase.

In some embodiments, the impedance spectrum resulting from a control sample and the impedance spectrum resulting from a test sample each comprise a feature at a first frequency and a second frequency respectively, and wherein the amount of analyte is determined by reference to the difference between the first frequency and the second frequency.

In some embodiments, the feature is a peak or a maximum, a trough or a minimum, an upwardly slanting portion of the spectrum, or a downwardly slanting portion of the spectrum.

In some embodiments, the step of using the impedance spectrum to determine the amount of analyte comprises using the spectrum to determine electron transfer kinetics between a redox reporter of the electrochemical sensor and a surface of the working electrode.

In some embodiments, the electron kinetics is an electron transfer rate between the redox reporter of the electrochemical sensor and the working electrode surface.

In some embodiments, the method is capable of repeatedly determining a concentration of an analyte at an interval of less than 1 min, 50 seconds, 40 seconds, 30 seconds, 20 seconds, 10 seconds, 9 seconds, 8 seconds, 7 seconds, 6 seconds, 5 seconds, 4 seconds, 3 seconds, 2 seconds, or 1 second.

In some embodiments, the method comprises repeatedly determining a concentration of an analyte at an interval of less than 1 min, 50 seconds, 40 seconds, 30 seconds, 20 seconds, 10 seconds, 9 seconds, 8 seconds, 7 seconds, 6 seconds, 5 seconds, 4 seconds, 3 seconds, 2 seconds, or 1 second.

In some embodiments, the method comprises repeatedly determining a concentration of an analyte for at least 1 hour, 2 hours, 3 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 30 hours, 36 hours, 42 hours, or 48 hours.

In some embodiments, the method does not require any frequency sweeping step across a frequency range to determine the amount of analyte in a sample.

In some embodiments, the method does not require any calibration step to determine the amount of analyte in a sample.

In some embodiments, the method does not require any drift adjustment step to determine the amount of analyte in a sample.

In some embodiments, the sample is a bodily fluid within or about the body of a subject.

In some embodiments, the bodily fluid is selected from: interstitial fluid (ISF), blood, saliva, a lacrimal secretion, a lactational secretion, a nasal secretion, a tracheal secretion, a bronchial secretion, an alveolar secretion, a gastric secretion, a gastric content, a glandular secretion, a vaginal secretion, a uterine secretion, a prostate secretion, semen, urine, sweat, cerebrospinal fluid, a glomerular filtrate, an hepatic secretion, bile, and an intraocular fluid.

In some embodiments, the working electrode is a wire, a needle, or a microneedle.

In some embodiments, the electrochemical sensor comprises a recognition element configured to specifically recognize a target analyte.

In some embodiments, the recognition element is associated with a redox reporter.

In some embodiments, the recognition element and/or the redox reporter undergo a change in the presence of the target analyte, the change altering a rate of electron transfer between the redox reporter and a surface of the working electrode.

In some embodiments, the change in the recognition element is a conformational change.

In some embodiments, the conformational change in the recognition element alters a distance between the redox reporter and a surface of the working electrode.

In some embodiments, the conformational change in the recognition element alters a reorganization energy of the redox reporter.

In some embodiments, a rate at which the redox reporter approaches a surface of the working electrode is altered in the presence of the target analyte.

In some embodiments, a fraction of time that the redox reporter is proximal to a surface of the working electrode rather than distal is altered in the presence of the target analyte.

In some embodiments, the alteration in rate or the fraction of time is associated with a target analyte associated alteration in a steric bulk parameter, a biomolecular rigidity parameter, an electrostatic parameter, or a hydrodynamic radius of the redox reporter.

In some embodiments, the change in the recognition elements and/or the redox reporter is a dissociation of the recognition element from the target analyte.

In some embodiments, the conformational change alters a rate of electron transfer between a redox reporter associated with the recognition element and a surface of the working electrode.

In some embodiments, the recognition element is associated with a surface of the working electrode, and the redox reporter is associated with the recognition element and the conformational change in the recognition element alters a distance between the redox reporter and the surface of the working electrode which in turn alters a rate of electron transfer between the redox reporter and the surface of the working electrode.

In some embodiments, the recognition element is a biological polymer.

In some embodiments, the biological polymer is a nucleic acid.

In some embodiments, the biological polymer is an aptamer.

In some embodiments, the electrochemical sensor is configured as a wearable device.

Some embodiments include apparatus for detecting an amount of an analyte in a test sample, the apparatus comprising: an electrochemical sensor having a working electrode and a counter electrode; a power source configured to apply a voltage perturbation to the working electrode, the voltage perturbation being a sum of two or more sinusoidal waveforms, each of the two or more sinusoidal waveforms being of a different frequency; a voltage and/or a current measuring circuit connected across the working electrode and the counter electrode; and a processor configured to integrally transform a measured voltage and/or current to generate an impedance spectrum, and to use the impedance spectrum to determine the amount of analyte in the test sample.

In some embodiments, the processor has access to program instructions configured to execute the method of any one of claims 1 to 42.

In some embodiments, the electrochemical sensor comprises a redox reporter, and the processor has access to program instructions configured to execute the method of certain embodiments.

In some embodiments, the working electrode is a wire, a needle or a microneedle.

In some embodiments, the electrochemical sensor comprises a recognition element configured to specifically recognize a target analyte.

In some embodiments, the recognition element is associated with a redox reporter.

In some embodiments, the recognition element and/or the redox reporter undergo a change in the presence of the target analyte, the change altering a rate of electron transfer between the redox reporter and a surface of the working electrode.

In some embodiments, the change in the recognition element is a conformational change.

In some embodiments, the conformational change in the recognition element alters a distance between the redox reporter and a surface of the working electrode or a coupling constant describing electron transfer through the recognition element.

In some embodiments, the conformational change in the recognition element alters a reorganization energy of the redox reporter.

In some embodiments, a rate at which or fraction of time during which the redox reporter approaches a surface of the working electrode is altered in the presence of the target analyte.

In some embodiments, a fraction of time that the redox reporter is proximal to a surface of the working electrode rather than distal is altered in the presence of the target analyte.

In some embodiments, the alteration in rate or the fraction of time is associated with a target analyte associated alteration in a steric bulk parameter, a biomolecular rigidity parameter, an electrostatic parameter, or a hydrodynamic radius of the redox reporter.

In some embodiments, the change in the recognition elements and/or the redox reporter is a dissociation of the recognition element from the target analyte.

In some embodiments, the conformational change in the recognition element alters a rate of electron transfer between a redox reporter associated with the recognition element and a surface of the working electrode.

In some embodiments, the recognition element is associated with a surface of the working electrode, and the redox reporter is associated with the recognition element and the conformational change in the recognition element alters a distance between the redox reporter and the surface of the working electrode which in turn alters a rate of electron transfer between the redox reporter and the surface of the working electrode.

In some embodiments, the recognition element is a biological or biomimetic polymer.

In some embodiments, the biological polymer is a nucleic acid.

In some embodiments, the biological polymer is an aptamer.

In some embodiments, the electrochemical sensor is configured as a wearable device.

In some embodiments, non-transitory computer-readable media comprising computer-executable program instructions configured to execute the method of many embodiments.

In some embodiments, the program instructions are provided by the non-transitory computer-readable media of certain embodiments.

Additional embodiments and features are set forth in part in the description that follows, and in part will become apparent to those skilled in the art upon examination of the specification or may be learned by the practice of the disclosure. A further understanding of the nature and advantages of the present disclosure may be realized by reference to the remaining portions of the specification and the drawings, which forms a part of this disclosure.

### BRIEF DESCRIPTION OF THE FIGURES

The description will be more fully understood with reference to the following figures, which are presented as embodiments of the invention and should not be construed as a complete recitation of the scope of the invention, wherein:

FIGS. 1A through 1E illustrate diagrammatically some of the mechanisms by which the binding of a target analyte can alter electron transfer rates in electrochemical biosensors in accordance with an embodiment. Among other mechanisms, interaction of target analyte with a redox-reporter-modified receptor can change the rate of electron transfer between the redox reporter and the working electrode surface via, as in FIG. 1A, a binding-induced change in the shape of the reporter, or, as in FIG. 1B, by blocking the approach of the redox reporter to the electrode, or, as in FIG. 1C, by changing the reorganization energy of the reporter, or as in FIG. 1D, changing the rigidity of the receptor or the coupling constant describing electron transfer through the receptor, or, as in FIG. 1E, changing the hydrodynamic or steric radius of the receptor/target complex.

FIG. 2 illustrates impedance measurement as applied to the sensors of FIG. 1A in accordance with an embodiment.

FIGS. 3A through 3D illustrate a series of graphs demonstrating that when applied to an EAB sensor, fast Fourier transform electrochemical impedance spectroscopy (FFT-EIS) is able to rapidly and monotonically correlate electrochemical impedance to the target molecular concentration in accordance with an embodiment. (A) The response of a vancomycin-detecting EAB sensor when immersed in whole bovine blood and challenged with increasing quantities of the target molecule. (B) Analysis of FFT-EIS data using equivalent circuit modelling. (C) Challenge of the EAB sensor with increasing concentrations of vancomycin decreases electron transfer resistance. (D) The electron transfer rate approximated using Equation 5. The resulting binding curve fits a Hill-Langmuir isotherm with a binding constant K_{D}=144±31 μM (the latter reflects estimated 95% confidence intervals).

FIGS. 4A through 4D illustrate FFT-EIS supports high-frequency, real-time measurements in vivo in accordance with an embodiment. (A) Diagram showing experimental placement of sensor electrodes in living rat. (B) Graph showing that by use of FFT-EIS, it was possible to measure impedance spectra every ~1.8 second. Some downward drift is evident (C) shows a graph fitting the spectra revealing that the drift shown at panel (A) is correlated with an immediate, steady increase in charge transfer resistance (R_{ct}) and a corresponding decrease in pseudocapacitance of the redox reporter (C_{ads}). Upon injection of 30 mg/kg vancomycin, however, only R_{ct} is responsive. (D) Calculation of kₑₜ reveals that this parameter is stable prior to drug infusion, indicating the intrinsic electron transfer rate constant of the unbound state of the aptamer is unaffected by whatever is causing R_{ct} and C_{ads} to drift.

FIGS. 5A through 5C illustrate a series of graphs demonstrating use of FFT-EIS to interrogate EAB sensors yields rapidly time-resolved molecular measurements in vivo in accordance with an embodiment. Experiments in three separate animals (panels A, B and C respectively) confirm the absence of the drug pre-infusion and show the expected concentration spike after infusion (the error bars reflect 95% confidence intervals).

FIGS. 6A and 6B illustrate a series of graphs demonstrating the use of FFT-EIS to interrogate phenylalanine-detecting EAB sensors and monitor that analyte in the jugular veins of live rats with 1.8 second resolution for a fasted animal (A) and non-fasted animal (B) in accordance with an embodiment. The fasted animal was infused with two sequential doses of phenylalanine and in both cases, the concentration of free phenylalanine in the blood quickly decayed back to the pre-infusion baseline. In contrast, for the non-fasted animal, return to the post-infusion baseline was slower as expected having regard to prior studies of phenylalanine homeostasis in such animals.

FIG. 7 shows a table detailing the frequencies, amplitudes, and phases used to construct the multi-frequency perturbation waveform as used in the Examples in accordance with an embodiment.

FIGS. 8A through 8C illustrate a series of sensor cyclic voltammograms of a vancomycin detecting EAB sensor in accordance with an embodiment. Cyclic voltammograms were recorded prior to all EIS experiments to determine E_{1/2}, which was applied as the DC bias during electrochemical impedance spectroscopy (EIS). (A) EAB sensor in 1×PBS plus 2 mM MgCl₂. The voltammogram was recorded at 25° C. with a scan rate of 100 mV/s. The half wave potential (E_{1/2}), being the mean voltage between the reductive and oxidative peak potentials was determined to be −285 mV versus Ag|AgCl. (B) EAB sensor in vivo, E_{1/2} (black dashed line) is −324 mV; the difference arising from the use of an anodized silver wire as the reference electrode. (C) EAB sensor in vivo after vancomycin dosing. The peak area is reduced (presumably due to monolayer loss), E_{1/2} remains at −324 mV.

FIGS. 9A and 9B illustrate graphs of (A) Frequency- and (B) time-domain representations of the multi-sine EIS waveform employed in the Examples in accordance with an embodiment.

FIG. 10 shows Bode |Z| plots from a vancomycin-detecting EAB sensor in blood at 37° C. upon challenge with increasing concentrations of vancomycin in accordance with an embodiment.

FIGS. 11A and 11B illustrate phase (blue) and modulus (green) Bode plots of a 10 kΩ resistor in accordance with an embodiment. (A) The spectrum without filter corrections showing non-zero phase at higher frequencies, and. (B) after correction, showing phase is zero across the frequency spectrum.

FIG. 12 shows a binding curve of a phenylalanine-detecting EAB sensor interrogated, demonstrating that in fresh rat's blood the present invention may be used to determine phenylalanine concentration from kₑₜ values derived from FFT-EIS. Values of kₑₜ (black points, error bars represent the standard deviation across four independently fabricated and tested sensors) fall on a Langmuir isotherm in accordance with an embodiment.

FIGS. 13A and 13B illustrate that FFT-EIS interrogation of a lactate-detecting EAB sensor supports the real-time monitoring of plasma lactate concentrations in a living rat in accordance with an embodiment. (A) Shown is calibration of the lactate-detecting sensor in undiluted bovine blood held at 37° C. using FFT-EIS interrogation. The lactate concentration was increased in blood from 6 mM to 106 mM prior to the calibration. During the calibration, the lactate concentration was lowered by diluting the blood with 106 mM lactate stepwise by adding blood without extra lactate and PBS+25 mg/mL BSA. The kₑₜ was measured after each dilution step. (B) FFT-EIS interrogation of the EAB sensor supports real time plasma lactate monitoring in vivo using the rat animal model. Prior to the intravenous infusion of 1000 mg/kg lactate, stable baseline concentrations were observed. The plasma concentration increases sharply upon intravenous infusion of lactate and returns afterwards back to its original level.

Unless otherwise indicated herein, features of the drawings labelled with the same numeral are taken to be the same features, or at least functionally similar features, when used across different drawings.

The drawings are not prepared to any particular scale or dimension and are not presented as being a completely accurate presentation of the various embodiments.

### DETAILED DESCRIPTION

After considering this description it will be apparent to one skilled in the art how the invention is implemented in various alternative embodiments and alternative applications. However, although various embodiments of the present invention will be described herein, it is understood that these embodiments are presented by way of example only, and not limitation. As such, this description of various alternative embodiments should not be construed to limit the scope or breadth of the present invention. Furthermore, statements of advantages or other aspects apply to specific exemplary embodiments, and not necessarily to all embodiments, or indeed any embodiment covered by the claims.

Throughout the description and the claims of this specification the word “comprise,” and variations of the word, such as “comprising” and “comprises,” is not intended to exclude other additives, components, integers, or steps.

Reference throughout this specification to “one embodiment” or “an embodiment” means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment, but may.

The present invention is predicated at least in part on the inventors' discovery that electrochemical impedance spectroscopy (EIS) utilizing measurements taken at a plurality of frequencies simultaneously may be used in an electrochemical sensor interrogation method. Such a method may obviate the need for any sensor calibration prior to use, and may also achieve superior time resolution.

In this approach, impedance (as determined using a voltage and/or current measured across the working electrode and the counter electrode) is measured simultaneously across a range of frequencies. At higher frequencies, impedance informs on rapid processes, such as the formation of the electrochemical double layer. Impedances measured at lower frequencies, in contrast, are typically associated with slower processes, such as electron transfer, adsorption and intercalation events, as well as mass transport. These lower frequencies are of particular interest given ability to follow changes in electron transfer between a sensor redox reporter and the sensor working electrode surface can be used to determine the amount of target analyte present in a test sample.

In some embodiments, a fast Fourier transform electrochemical impedance spectroscopy (FFT-EIS) is used to simultaneously measure impedance at multiple frequencies, yielding both high time resolution and the depth of information contained in a full impedance spectrum. In this way, it is possible to estimate kₑₜ (electron transfer kinetics), and from that information to determine target analyte concentration. Concentration may be output very rapidly (e.g., every few seconds), providing a method of interrogating electrochemical sensors that is ideally both more-highly-time-resolved and calibration free.

In many embodiments, an exemplary electrochemical sensor is an EAB sensor. These sensors are capable of measuring the concentrations of specific target analytes in the body in real time and may revolutionize the monitoring of health and the diagnosis and treatment of disease. By providing a real-time window into drug and biomarker concentrations in plasma, in interstitial fluid, or in another bodily fluid, for example, EAB sensors may significantly improve the individualization of pharmacological treatments.

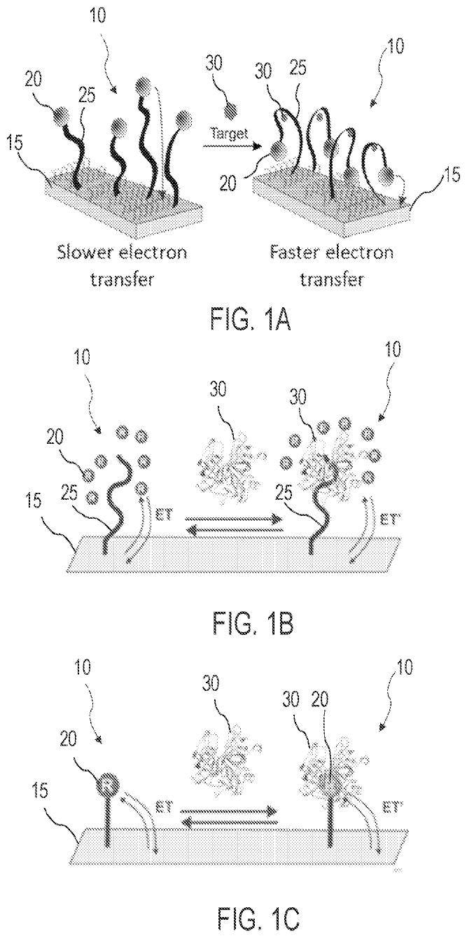

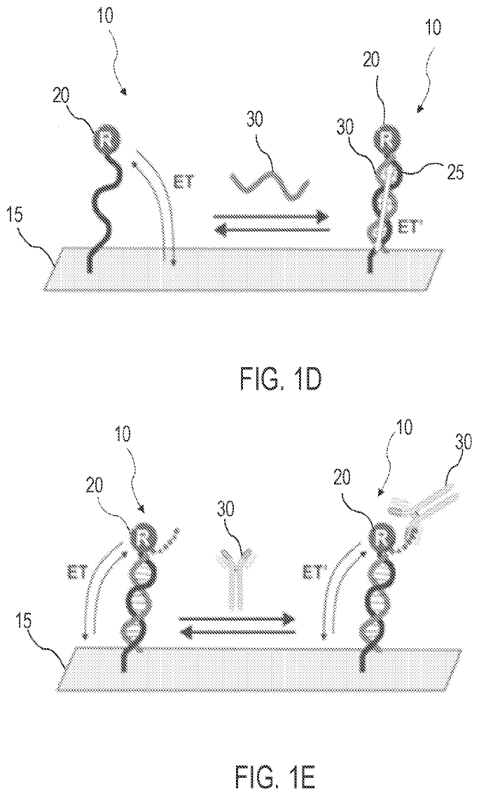

Reference is made to FIG. 1A illustrating an EAB sensor (10) comprised of a gold electrode (15) on which a sub-monolayer of target-recognizing, redox reporter (20)-modified, nucleic acid aptamers (25) are deposited via thiol-on-gold self-assembled monolayer formation. In some embodiments, introduction of the target analyte (30) triggers a conformational change in the aptamer (25), altering the rate of electron transfer (kₑₜ) between the redox reporter (20) and the electrode (15). In some embodiments, binding induced displacement of the redox reporter from the binding pocket could also play a role.

The change in kₑₜ informs on the target analyte concentration in real time without the addition of exogenous reagents. Of note, this signal transduction mechanism does not rely on the chemical transformation of the target, rendering the approach general. Consistent with this, EAB sensors have been used for real-time measurement of multiple drugs, metabolites, neurotransmitters, hormones, toxins, and protein biomarkers both in vitro and in vivo.

EAB sensors have been used in many non-clinical applications such as environmental monitoring and for monitoring manufacturing processes.

It will be understood that the present invention is applicable to electrochemical sensors other than EAB sensors. It is proposed that the invention may be operable for any electrochemical sensor type that relies on a change in electron kinetics in the detection of a target analyte. Examples include the sensor (10) illustrated in FIG. 1B based on binding-induced changes linked to changes in the diffusion of solution-phase redox-reporter (20), resulting in an altered rate of electron transfer (ET to ET′).

A further example is illustrated in FIG. 1C being a sensor (10) based on binding-induced displacement of ligands on the reporter (20) or binding-induced changes in redox-reporter's reorganizational energy.

The example at FIG. 1D is a sensor (10) based on binding-induced changes in the flexibility of a reporter or on the coupling constant that defines how rapidly electrons can transfer between the electrode (15) and the reporter (20).

The further example illustrated in FIG. 1E is a sensor (10) based on sterically or hydrodynamically induced changes in the efficiency with which a scaffold-attached redox reporter (20) approaches an underlying electrode (15) surface.

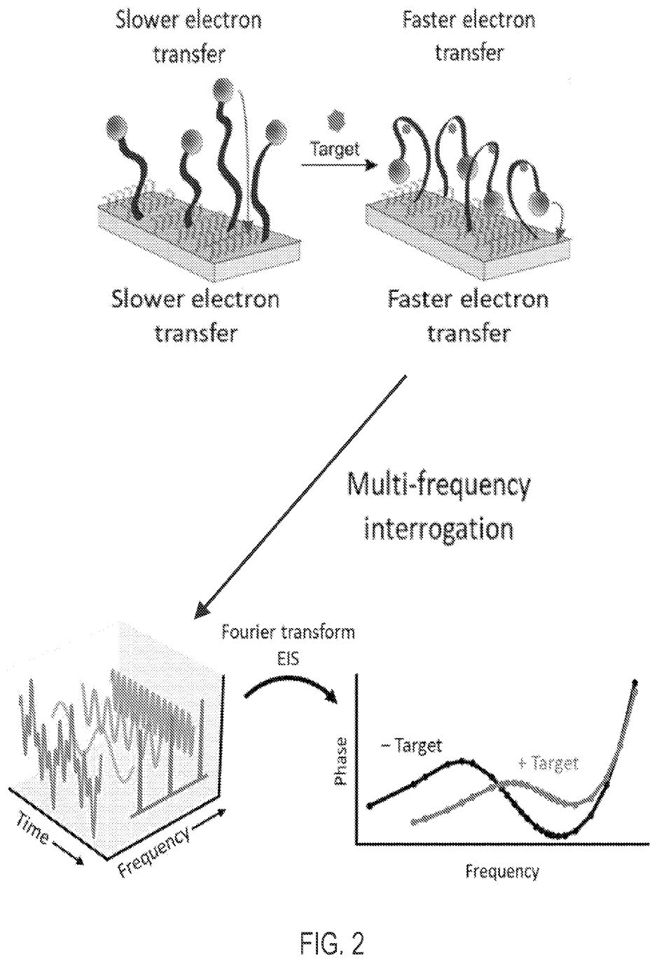

The present invention is further described by reference to the scheme illustrated in FIG. 2, using an EAB sensor as exemplary form of sensor only. The sensor working electrode has a DC voltage applied thereto, with a voltage perturbation being applied to the DC voltage. The voltage perturbation is the sum of a plurality of sinusoidal waveforms, each of which is of a different frequency. The sensor current (shown as the left-most trace in the lower left panel) is subjected to FFT-EIS to simultaneously measure impedance at multiple frequencies. The lower right-hand panel shows a graph of phase as a function of frequency, with a phase shift being evident in the presence of target analyte. The simultaneous nature of the impedance measure avoids any need to sweep multiple frequencies allowing for readings to be taken in rapid succession thereby improving time resolution. Furthermore, the electron transfer rate and thus the sensor response is independent of the number of functional aptamers on the working electrode surface, and therefore the need to calibrate a sensor is avoided.

In EIS, a sinusoidal oscillating voltage on top of a set DC bias is applied to the working electrode and the (sinusoidal) current response is recorded. The impedance, Z, at a particular frequency ω is defined as the ratio between voltage and current at that frequency (Equation 1), with the “lag” between the voltage perturbation and the current response quantified as the phase shift φ.

$$Z ( ω ) = \frac{\vert V \vert sin ( ω t )}{\vert I \vert sin ( ω t + ϕ )} = \vert Z \vert {e}^{i ϕ} \qquad ( 1 )$$

Here, |V| and |I| are the amplitudes of the voltage and current, respectively, w is the frequency, i is the square root of −1, and |Z| is the magnitude of the impedance. The primary benefit of EIS (and its label of “spectroscopy”) arises from the measurement of Z across a wide range of frequencies (e.g., millihertz to kilohertz). Specifically, frequency-dependent impedance measurements can be used to characterize processes ranging from the rapid charging of the electric double layer at high frequencies to electron transfer reactions and molecular diffusion occurring on much longer time scales.

In applications such as the real-time measurement of specific molecules in the living body, a limitation of EIS is that its time resolution is typically poor. Specifically, with traditional, “frequency-sweep” EIS, each frequency, f, interrogated requires at least 1/f measurement time. Given this, the measurement of spectra down to frequencies of the order 1 Hz requires total acquisition times of tens of seconds or more. In the present invention, FFT-EIS retains the information contained in the full frequency range while significantly decreasing acquisition time. It does so by measuring the impedance at many frequencies simultaneously.

In one use of FFT-EIS according to the present invention, the applied voltage perturbation is a superposition of 18 sine waves spanning the desired frequency range. It will be appreciated that other numbers of sine waves may be operable, such as at least about 5, 10, 15, 20, 25, 30, 35, 40, 45, or 50 sine waves.

Due to the approximate linearity of electrochemical systems over small voltage changes, the resulting current response is a superposition of the current response at each of the applied frequencies. A fast Fourier transform of the recorded voltage and current data thus yields an impedance spectrum. Using this approach, a complete impedance spectrum may be collected in the time scale defined by the slowest applied frequency (t_{spectrum}≥1/fₘᵢₙ).

Electron transfer kinetics can be extracted from impedance spectra by, for example, first fitting the spectra to an equivalent circuit to extract the parameters describing the circuit, such as the adsorption pseudocapacitance C_{ad}, the solution resistance, Rₛ, the double layer capacitance, C_{dl}, and the charge transfer resistance, R_{ct}. Electron transfer kinetics can then be estimated from its relationship to these parameters.

The present invention will now be more fully described by reference to the following non-limiting Examples.

### Example 1: Materials and Methods Used in Experimental Work Described in Examples 2 Through 6

### Example 1.1 Materials

In vitro sensors were made using 0.2 mm diameter gold wire (99.99% purity) insulated with polyolefin heat-shrink tubing (0.05″, 0.017″, 0.007″). For in vitro tests, a commercial Ag|AgCl(s) reference electrode (and a commercial platinum reference electrode) was used. However, it is noted that such tests can be performed with any reference electrode that is suitable for in vitro and in vivo experiments. Intravenous sensors used for in vivo measurements were made using 0.2 mm diameter gold wire, 0.005 in. diameter platinum wire (99.99% purity) and 0.005 in. diameter silver wire (99.99% purity). The insulation used for these sensors was polytetrafluoroethylene heat-shrink (HS Sub-Lite-Wall, 0.02, 0.005, 0.003±0.001 in, black) Sodium hydroxide, 6-mercapto-1-hexanol, Tris (2-carboxyethyl) phosphine, sulfuric acid, phenylalanine, and the phenylalanine assay kit were obtained. Phosphate buffered saline (PBS) was diluted from a 20× stock. Vancomycin-HCl and Methylene blue- and HO—C₆S—S—C₆-modified DNA sequences were purchased from commercially available resources; their sequences are listed infra.

DNA sequences employed

| Name | Sequence and modifications |
|---|---|
| Vancomycin | HS-C₆-CGAGGGTACCGCAATAGTACTTATTGTT |
| | CGCCTATTGTGGGTCGG-MB |
| Phenylalanine | HS-C₆-CG ACC GCG TTT CCC AAG AAA |
| | GCA AGT ATT GGT TGG TCG-MB |

### Example 1.2 Sensor Fabrication

In vitro sensors were made by shrink wrapping gold wire with polyolefin and leaving 3 mm of the wire exposed. These sensors were made ahead of time and required no additional steps prior to electrochemical cleaning.

In vivo sensors for intravenous use were fabricated from wires as follows: gold (for the working electrode), platinum (for the counter electrode) and silver (for the reference electrode). These wires were individually insulated with polytetrafluoroethylene heat-shrink and bundled together in a staggered manner with the gold wire at the bottom, followed by the platinum and then the silver wire. The exposed lengths of each wire were 3 mm, 6 mm, and 1 cm, respectively. Once bundled together, the intravenous, three-electrode sensors were immersed overnight in household bleach (sodium hypochlorite 7.5%) to chlorinate the silver electrode. The three electrodes were subsequently rinsed with Millipore water prior to electrochemical cleaning.

Prior to aptamer deposition, the gold working electrode was electrochemically cleaned in NaOH followed by roughening in H₂SO₄ using a CH1040C potentiostat. The cleaning involved cycling the potential between −1.0 V and −2 V at 2 V/s 1000 times while the electrodes were immersed in 0.5 M NaOH. This was followed by roughening in 0.5 M H₂SO₄ with the application of 20 ms pulses at 0 V and 2.2 V 32000 times as previously done to increase the electrode's microscopic surface area. The electrodes were subsequently analyzed by cyclic voltammetry in 0.5 M H₂SO₄ (between 1.5 and −0.35 V at 1 V/s) to determine their electroactive surface area. Intravenous sensors to be used in vivo were inserted into a 20G catheter at this point.

To functionalize the working electrode the disulfide bond was first reduced in the stock alkanethiol-and-methylene-blue modified aptamer by combining 14 μL of 10 mM tris (2-carboxyethyl) phosphine with 2 μL of 100 μM DNA for 1 h in the dark. The gold electrodes were electrochemically cleaned and roughened with Millipore water and followed by immersion for 1 h in 500 nM reduced DNA in PBS. The electrodes were then transferred to a 10 mM solution of 6-mercapto-1-hexanol in PBS and stored overnight before use.

### Example 1.3 Electrochemical Measurements

All electrochemical measurements were carried out using a three-electrode setup. In vitro experiments employed an Ag|AgCl (saturated KCl) reference electrode and a platinum wire counter electrode. In vivo experiments used a silver wire coated with silver chloride (as described above) as the reference electrode and the platinum wire counter electrode. As will be appreciated, any other reference electrode suitable for in vivo use may substitute.

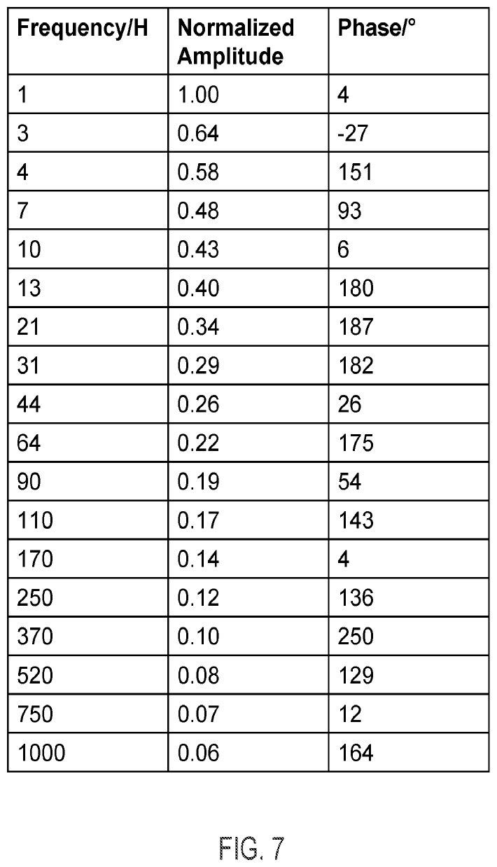

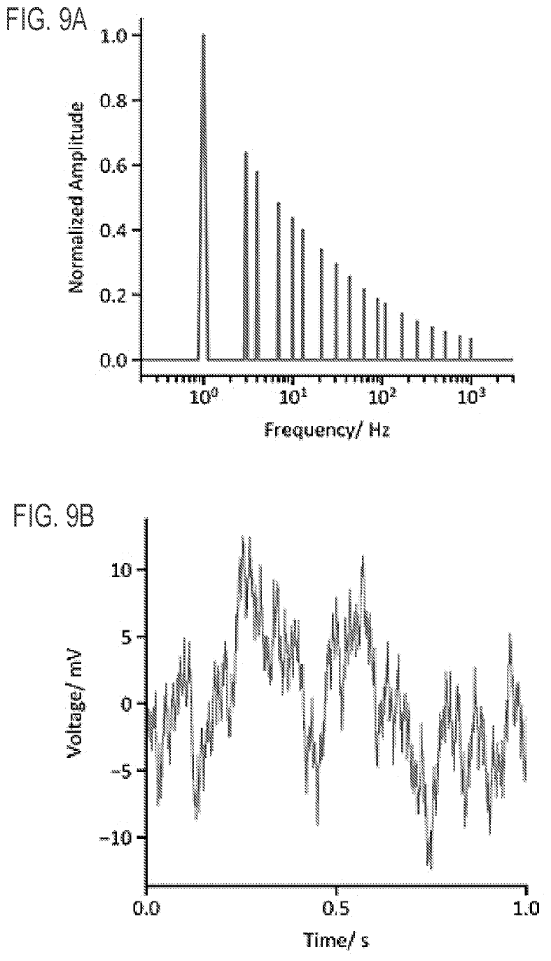

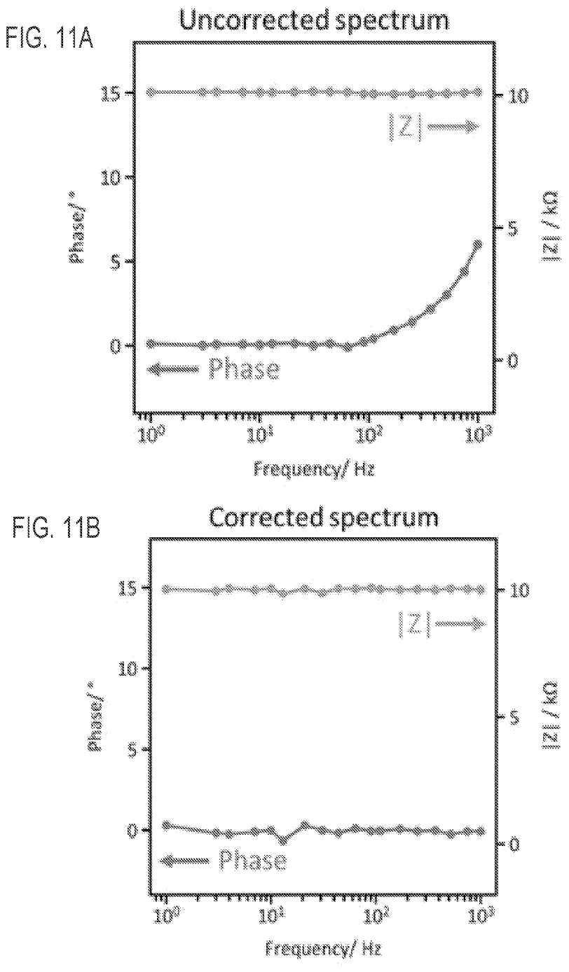

All electrochemical measurements were performed using an Autolab PGStat128N. The potentiostat was configured in “high stability mode” with a current range of ±1 μA, which affects the filter characteristics. For FFT-EIS measurements, the multi-sin waveform was generated by a DG812 arbitrary waveform generator (Rigol Technologies) and fed into the potentiostat's external voltage input using a BNC connection. The voltage waveform consisted of a superposition of 18 sine waves at logarithmically spaced frequencies ranging from 1 Hz to 1 kHz. The amplitude and phase of each sinusoidal oscillation were optimized for maximum signal-to-noise as discussed in the supporting information of Example 7, and the summed waveform was scaled to have a peak-to-peak amplitude of 25 mV. The potentiostat's native software (NOVA) was used to set the DC bias—the formal potential of methylene blue, as measured by cyclic voltammetry—on top of the AC perturbation. Voltage and current were recorded at 70 kHz using an SDS1202X-E oscilloscope. After each oscilloscope frame (1.4 s) was collected, the current and voltage data were transferred to the host computer, fast Fourier transformed, saved, and displayed on a GUI for real-time monitoring. Data recording and processing were controlled by a custom Python program. Further details on the chosen waveform and artefact correction are described in supporting information of Example 7 (FIG. 7, FIG. 9, FIG. 11).

Impedance spectra were fitted to the equivalent circuit model using MEISP 3.0 after each experiment was complete. The adsorption pseudocapacitance C_{ads} was modelled as a constant phase element, given by Equation 2 (i is the imaginary number, ω is frequency, and n is the constant phase parameter). The parameter n was fixed at 0.84 for all fits to improve consistency in the fitted C_{ads} values.

$${Z}_{{C}_{ads}} ( ω ) = \frac{1}{{{C}_{ads} ( i ω )}^{n \cdot 90 °}} \qquad ( 2 )$$

### Example 1.4 In Vivo Measurements

All in vivo experiments were performed on male Sprague-Dawley rats (4-5 months old). The rats weighed between 350-500 g and were pair-housed in a standard light cycle room (12:12 regular light cycle with lights on at 8 AM). They were allowed ad libitum access to food and water and the Institutional Animal Care and Use Committee (IACUC) of the University of California at Santa Barbara approved the experimental protocol which adhered to the guidelines given by the NIH Guide for Care and Use of Laboratory Animals.

Prior to the measurement, the rats were anesthetized using 4% isofluorane in a Plexiglas anesthesia chamber. Anesthesia was then maintained via a nose cone for the entire duration of the experiment at a level of 2-3% isofluorane. The neck was shaved and dissected in order to surgically isolate the left and right jugular veins. After isolating the two jugular veins, each one was tied off using sterile 6-0 silk sutures. Prior to the measurement, the wires in the sensor were adjusted such that the counter and working electrode were exposed outside of the 20G catheter into the vein as previously described. A small incision was then made in each vein using spring-loaded microscissors that allowed us to insert the sensor-containing catheter into the right jugular vein and an infusion line into the left jugular vein. Both the sensor and drug infusion catheter were anchored in place using two sterile 6-0 silk sutures. 30 units of heparin were infused through the infusion line immediately after insertion of the sensor and prior to any recordings. To intravenously dose the rats at 30 mg/kg a precalculated volume of 0.05 M vancomycin solution was injected using a syringe pump.

### Example 2: FFT-EIS can Rapidly Relate Electrochemical Impedance to the Target Molecular Concentration in an EAB Sensor

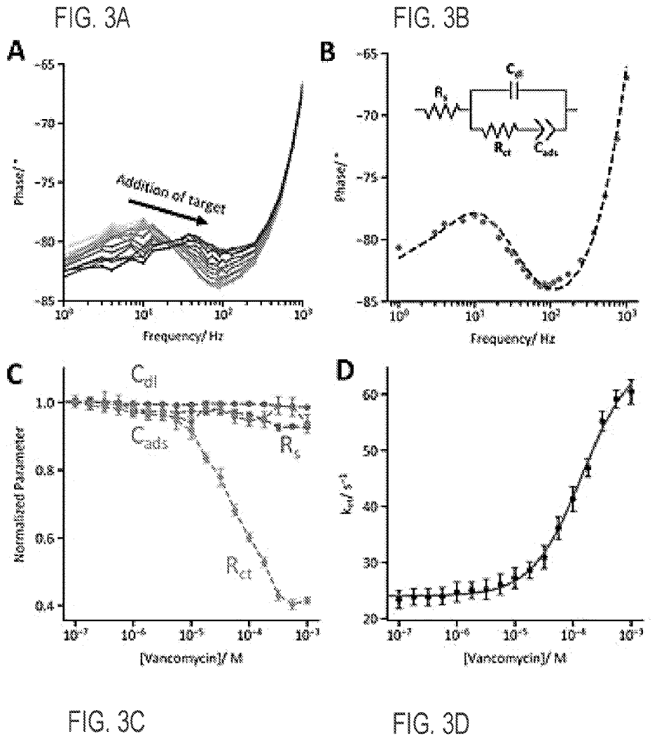

Reference is made to FIG. 3. In panel (A) the vancomycin-detecting EAB sensor was immersed in whole bovine blood at 37° C. and challenged with increasing quantities of the target molecule. This induced conformational change in the aptamer which caused a shift in the impedance spectrum, as resolved by FFT-EIS.

In panel (B) FFT-EIS data was analyzed using equivalent circuit modelling. The circuit model employed comprises of resistors representing the bulk solution resistance (Rₛ) and the Faradaic electron transfer between the electrode and the methylene blue moieties (R_{ct}), as well as capacitors representing the electrochemical double layer (C_{dl}) and the pseudocapacitance between the electrode surface and the surface-bound methylene blue (C_{ads}). The transfer function of this circuit was calculated using Kirchhoff's laws and experimentally measured data (blue points) were fit to this function (dotted line). The data presented here were collected from a vancomycin-detecting sensor immersed in whole bovine blood at 37° C. in the absence of vancomycin.

In panel (C), challenging the EAB sensor with increasing concentrations of its vancomycin target reveals that increasing target concentration predominantly impacts (here, decreases) R_{ct}. This is because a low electron transfer resistance corresponds to a high electron transfer rate constant, corresponding to the target-bound state of the aptamer (in this figure the error bars represent standard deviations across four independently fabricated and interrogated sensors).

In panel (D) the electron transfer rate kₑₜ can be approximated from R_{ct} and C_{ads} using Equation 5. The resulting binding curve fits a Hill-Langmuir isotherm with a dissociation constant (K_{D}) of 144±31 μM (the latter reflects estimated 95% confidence intervals).

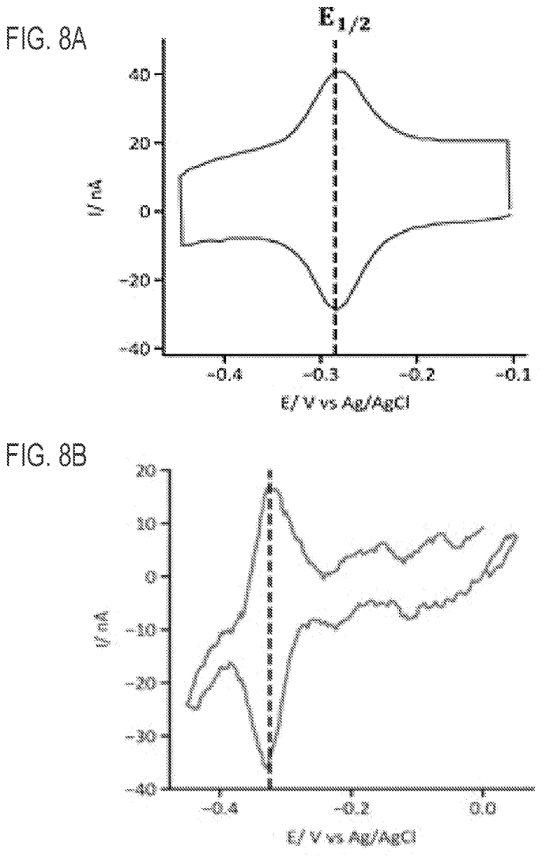

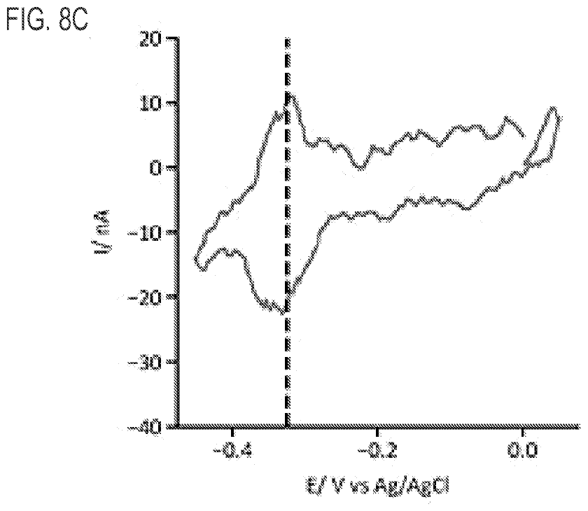

The impedimetric properties of EAB sensors, which are sensitive to target concentration, can be rapidly measured using FFT-EIS to enable highly-time resolved molecular measurements. To demonstrate this, the half-wave potential (E_{1/2}) of the sensor's methylene blue redox reporter was applied as the DC bias (prior to each experiment and E_{1/2} was determined which is typically around-0.285 V versus Ag|AgCl, using cyclic voltammetry; FIG. 8). FFT-EIS was then used to record impedance spectra as the sensor was immersed in whole bovine blood at 37° C. When the spectrum obtained in the absence of target molecule is displayed as a Bode plot (phase versus frequency; FIG. 3A, black trace), a local maximum is observed around 10 Hz, reflecting the rate of electron transfer between the methylene blue and the electrode surface. As expected, (given that the rate of electron transfer between the methylene blue and the electrode increases upon target binding), this peak steadily shifts to higher frequencies when the sensor is exposed to increasing concentrations of vancomycin. To determine the origin of this concentration-dependent shift, equivalent circuit modelling was applied using a simple, four-element equivalent circuit that has previously been used to represent the surface-tethered redox species seen in EAB sensors (FIG. 3B). This equivalent circuit includes a resistor, modeling bulk solution resistance (Rₛ), that is in series with the three other components: a capacitor, representing interfacial double-layer capacitance (C_{dl}), in parallel with a resistor, representing the Faradaic charge transfer resistance (R_{ct}), and a capacitor (C_{ads}), representing the surface-attached methylene blues. To better account for the rough, non-ideal surface of the EAB sensor, the latter is modelled as a constant phase element, rather than a true capacitor. This model fits the FFT-EIS data quite well (χ²~0.015, FIG. 3, panel B), suggesting that this four-component equivalent circuit is an adequate description of the physics of the sensor.

The components of the equivalent circuit behave as expected in response to the sensor's being challenged with its target. Rₛ, C_{dl}, and C_{ads}, for example, are effectively independent of vancomycin concentration. R_{ct}, in contrast, decreases with increasing vancomycin concentration (FIG. 3, panel C). This presumably arises due to the increased rate of electron transfer between the electrode and the bound, folded aptamer, as R_{ct} is inversely proportional to kₑₜ:

$${R}_{c t} = \frac{2 R T}{{F}^{2} A Γ {k}_{e t}} \qquad ( 3 )$$

Here, R is the gas constant, T is temperature, F is Faraday's constant, A is the electrochemical surface area of the working electrode, and Γ is the surface coverage of the redox-active molecule. Other than kₑₜ, each of these variables are constant during a given experiment, and thus the decrease in R_{ct} is entirely attributed to an increase in kₑₜ. Since, in turn C_{ads} is given by:

$${C}_{ads} = \frac{{F}^{2} A Γ}{4 R T} \qquad ( 4 )$$

kₑₜ can be calculated from the R_{ct} and C_{ads} as

$${k}_{et} = \frac{1}{2 {R}_{ct} {C}_{ads}} \qquad ( 5 )$$

Given that the rate of electron transfer from the redox reporter is dependent on whether the aptamer is target bound, kₑₜ should trace a Langmuir-Hill isotherm when plotted versus vancomycin concentration. As expected, it does (FIG. 3, panel D). The resulting monotonic relationship can be used to convert kₑₜ into estimates of vancomycin concentration in a manner that is calibration free. Specifically, kₑₜ is independent of the number of surface-bound, methylene-blue-modified aptamers and thus is independent of important sensor-to-sensor sources of fabrication variability, such as changes in the microscopic surface area of the electrode, or changes in the aptamer packing density, which would change the number of methylene-blue-modified aptamers and thus the absolute Faradaic current. Because of this, a kₑₜ versus vancomycin concentration calibration curve measured for a single sensor can be applied to all other sensors utilizing the same aptamer, obviating the need to calibrate each individual sensor.

### Example 3: FFT-EIS Supports High-Frequency, Real-Time Measurements in the Living Body

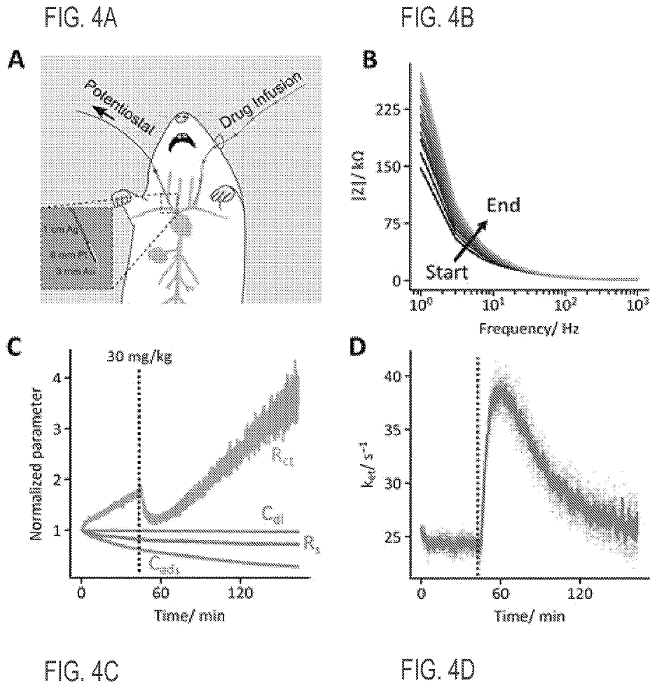

Reference is made to FIG. 4. In panel (A) in vivo EAB sensors are comprised of an aptamer-functionalized gold working electrode, a platinum counter electrode, and an Ag|AgCl reference electrode, each bound in heat-shrink tubing and inserted via a catheter into the right jugular vein of an anesthetized rat. Panel (B) Using FFT-EIS, impedance spectra were measured every ~1.8 second by applying the formal potential of methylene blue as the DC bias and the necessary multi-sine waveform as an AC perturbation. The magnitude of the impedance, |Z|, increased over the duration of the experiment, particularly at low frequencies. Presumably, this is due to fouling caused by nonspecific adsorption of proteins, cells, or small molecules to the sensor surface. Reference is made to Panel (C). Fitting the spectra reveals that this drift is correlated with an immediate, steady increase in R_{ct} and a corresponding decrease in C_{ads}. Upon injection of 30 mg/kg vancomycin, however, only R_{ct} is responsive. Reference is made to panel (D). Calculation of kₑₜ reveals that this parameter is stable prior to drug infusion, indicating the intrinsic electron transfer rate constant of the unbound state of the aptamer is unaffected by whatever is causing R_{ct} and C_{ads} to drift. Upon drug infusion, however, kₑₜ rises suddenly, indicating a larger population of the target-bound state of the aptamer. After the infusion is concluded, kₑₜ falls as the drug is excreted by the kidneys and the unbound aptamer again dominates. Here, the raw data (light blue points) are smoothed using a 13-s rolling average (dark blue trace).

### Example 4: FFT-EIS Interrogation of EAB Sensors Supports the Rapid Measurement of Specific Molecules in the Living Body

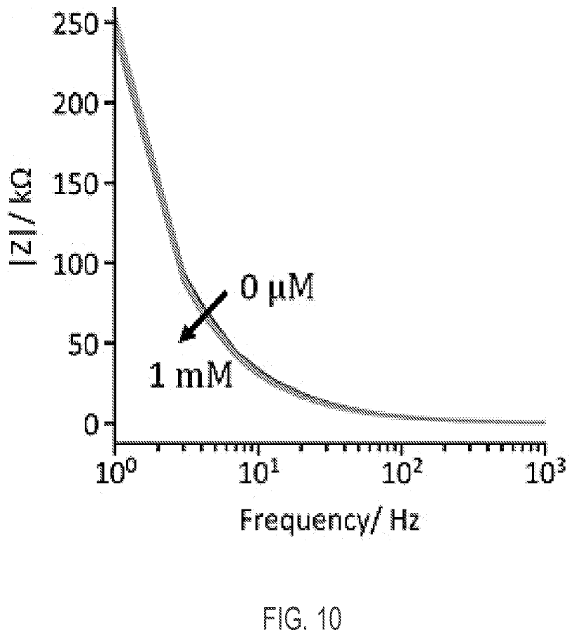

Aptamer-modified gold wire working electrodes were bundled with platinum counter and silver-silver chloride reference electrodes in a 20-gauge catheter (FIG. 4, panel A), and surgically inserted the resulting three-electrode sensor into the right jugular vein of an anesthetized rat. Full impedance spectra (containing 18 frequencies between 1 Hz and 1 kHz, FIG. 9) was then measured every 1.8 second for 2.5 hours. In contrast to the relatively unchanging impedance observed in whole blood in vitro (FIG. 10), the magnitude of the impedance increases over time under these in vivo conditions (FIG. 4, panel B), suggesting that the mechanisms by which EAB sensors degrade may differ between the two conditions. Fitting the time-resolved spectra indicates that this change in impedance is associated with a steady increase in R_{ct} and a corresponding decrease in C_{ads} (FIG. 4, panel C). This presumably occurs due to the nonspecific adsorption of proteins and cells to the electrode, which is expected will increase R_{ct} and decrease C_{ads} by reducing the number of methylene blue reporters that can access the electrode surface. In contrast, kₑₜ does not drift (FIG. 4, panel D), indicating that whatever causes impedance to drift does not affect the electron transfer kinetics of the aptamers that remain electrochemically accessible. Upon the infusion of 30 mg/kg vancomycin, however, kₑₜ rapidly rises as the aptamer shifts to its target-bound conformation (FIG. 4, panel D). Following the end of the infusion, kₑₜ returns to its baseline value as the drug is removed from the plasma via the kidneys.

### Example 5: Use of FFT-EIS to Interrogate EAB Sensors Yields Rapidly Time-Resolved Molecular Measurements in the Living Body

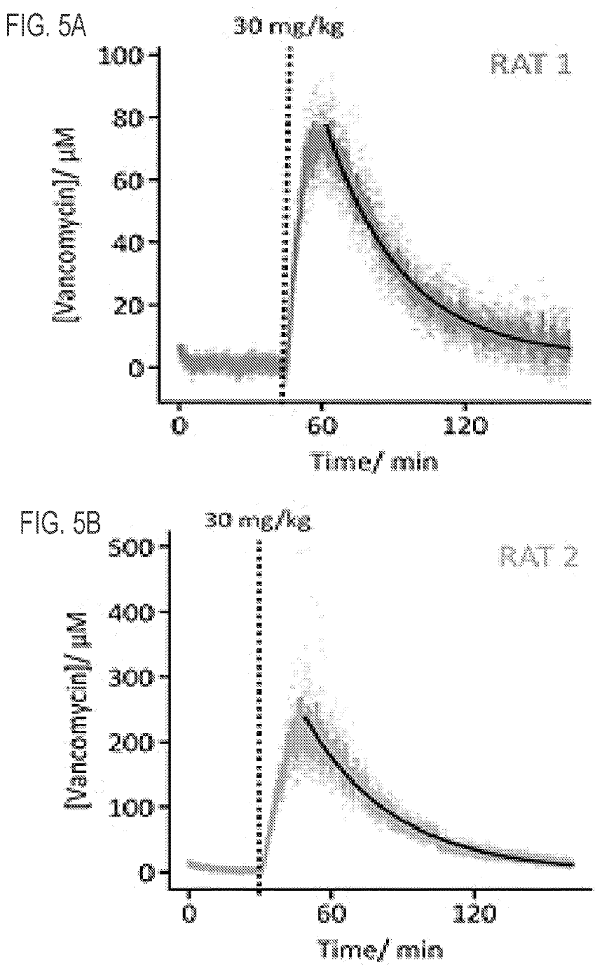

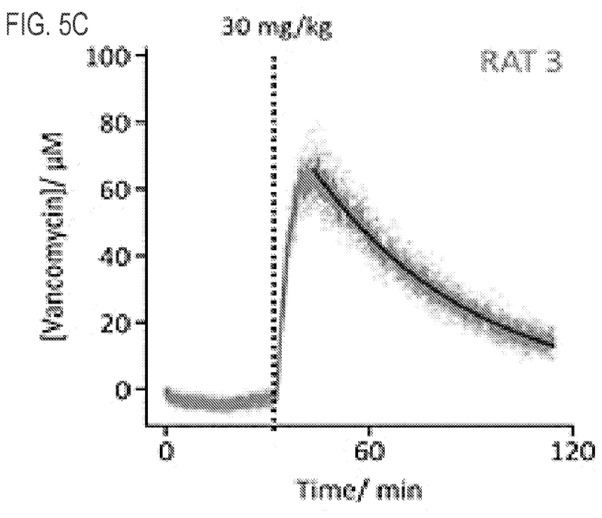

Reference is made to FIG. 5. Experiments in three separate animals confirm the absence of the drug pre-infusion, and show the expected concentration spike. Here, the raw data (light points) are smoothed using a 13-s rolling average (darker trace). The antibiotic concentration decayed monoexponentially (fits shown as black traces) after each dosing, with time constants of 33.1±0.5, 37.3±0.7, and 47.4±1.3 min (the error bars reflect 95% confidence intervals).

FFT-EIS interrogation of EAB sensors provides a highly-time-resolved window into molecular physiology and pharmacokinetics (FIG. 5, panel A). To see this, sensors were placed into the jugular veins of three rats. Prior to drug infusion, vancomycin concentrations were measured fluctuating tightly around zero (1.1±1.7 μM). Following infusion of the drug, its concentration was observed to rise to maxima of 70 to 250 UM before then falling exponentially with a time constants of 32 to 47 min. The different decay rates observed between three independent animals reflect each animal's unique physiology, highlighting the benefit of using EAB sensors to individualize clinical dosing.

### Example 6: Application of FFT-EIS to an EAB Sensor for the Detection of Phenylalanine

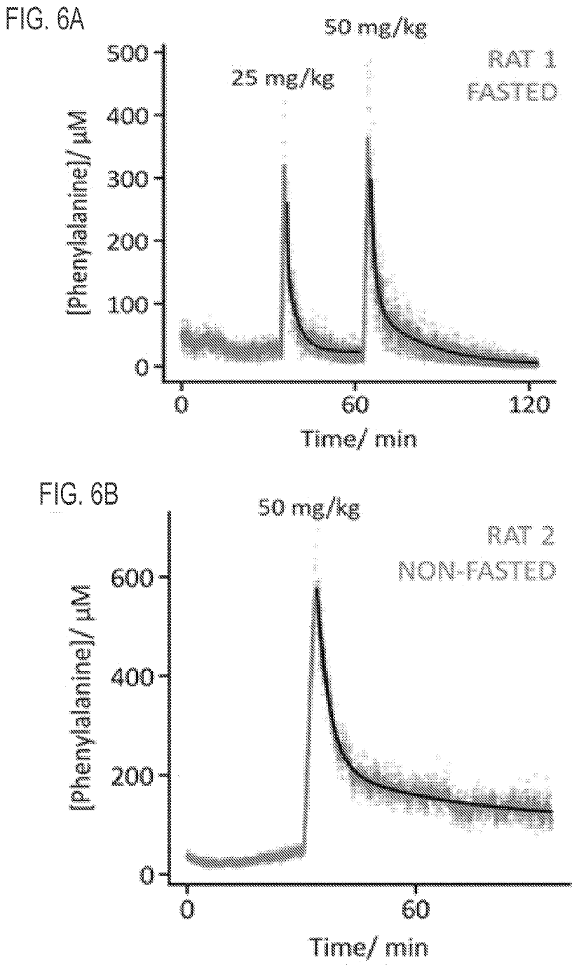

To demonstrate the general applicability of FFT-EIS as an EAB sensor interrogation technique, that technique was next employed to interrogate a sensor against the endogenous target phenylalanine (FIG. 6). Specifically, after calibrating a phenylalanine-detecting EAB sensor in vitro, FFT-EIS interrogation was used to measure the molecule's concentration in the jugular veins of anesthetized rats with 1.8 s resolution. Doing so, it was observed baseline phenylalanine concentrations of 41±10 μM (the latter reflects one standard deviation) in a fasted animal and 39±6 μM in a non-fasted animal, values in line with previous reports. Upon two intravenous infusions of additional phenylalanine, rapid rises to peak concentrations of 300-400 μM were observed, followed by rapid decays back to baseline in the fasted animal. Fitting the decay transients to the previously-reported biexponential model of phenylalanine kinetics yielded time constants of τ₁=0.2±0.1 minute and τ₂=3.8±0.2 minute for the first injection, and τ₁=1.7±0.1 minute and τ₂=20±2 minute for the second (error bars are 95% confidence intervals), suggesting that the animal's ability to rapidly store additional phenylalanine may have been saturated after the first challenge. In a non-fasted animal, in contrast, measurements showed a higher peak concentration (~600 M) and a slower decay (τ₁=3.3±0.1 minute and T₂=35±7 minute) to a higher, slowly decaying baseline following phenylalanine challenge, discrepancies that align with previous, in vivo measurements of phenylalanine kinetics in fasted and non-fasted rats.

Reference is made to FIG. 6. Using FFT-EIS to interrogate phenylalanine-detecting EAB sensors this metabolite has been monitored in situ in the jugulars of live rats with 1.8 s resolution. Reference Is made to panel (A). A fasted animal was infused with two sequential doses of phenylalanine. In both cases, the concentration of free phenylalanine in the blood quickly decayed back to the pre-infusion baseline (41±10 μM, 31±9 μM, and 25±10 μM before injection, after the first injection, and after the final injection, respectively). Reference is made to Panel (B). In contrast, the return to the post-infusion baseline was slower in a non-fasted animal, which is consistent with previous reports regarding phenylalanine homeostasis. Here, the raw data (light points) are smoothed using a 13-s rolling average (darker trace). Concentration decay transients were fitted to a two compartment (i.e., biexponential) model (black traces).

### Example 7: Supporting Information Relating to the Experimental Work Described in Examples 2 Through 6

### Example 7.1 Multi-Frequency Waveform

The Table shown at FIG. 7 details the frequencies, amplitudes, and phases used to construct the multi-frequency perturbation waveform.

### Example 7.2 Sensor Cyclic Voltammograms

Reference is made to FIG. 8. Panel (A); cyclic voltammogram recorded from a vancomycin-detecting EAB sensor in 1×PBS plus 2 mM MgCl₂. The voltammogram was recorded at 25° C. with a scan rate of 100 mV/s. The half wave potential (the mean voltage between the reductive and oxidative peak potentials), E_{1/2}, was determined to be −285 mV versus Ag|AgCl. Cyclic voltammograms were recorded prior to all EIS experiments in order to determine E_{1/2}¬, which was applied as the DC bias during EIS. Reference is made to panel (B). In vivo, E_{1/2} (black dashed line) is −324 mV; this difference arises from the use of an anodized silver wire as the reference electrode rather than the fritted, single junction reference electrode used in vitro. Reference is made to Panel (C). Shown is a cyclic voltammogram recorded in vivo at the end of a vancomycin dosing experiment. While the peak area is reduced (presumably due to monolayer loss), E_{1/2} remains at −324 mV.

Reference is made to FIG. 9. Panel (A) shows frequency- and panel (B) time-domain representations of the multi-sine EIS waveform employed.

A set of 18 logarithmically-spaced frequencies were chosen which were integer multiples of the fundamental frequency (1 Hz), avoiding any second harmonics. Phases were chosen in order to minimize constructive interference. Amplitudes were set to create a similar current output at every frequency (i.e., V(ω)∝|Z|(ω)). This strategy significantly increases signal-to-noise by applying higher voltage amplitudes at low frequencies, where (in an electrochemical cell) current is typically lower than at high frequencies. Using the frequencies f, amplitudes a, and phases φ listed (FIG. 7), the waveform was digitally synthesized as:

$$v ( t ) = {\sum }_{j} {a}_{j} sin ( 2 π {f}_{j} t + {ϕ}_{j} ) \qquad ( S1 )$$

The optimized waveform used in this study is shown in the frequency domain in FIG. 9 panel (A) and in the time domain in FIG. 9 (panel B). In the time domain, the peak-to-peak amplitude of the summed waveform was set to be 25 mV. The digital waveform was saved to a Rigol DG812 arbitrary waveform generator, which output the waveform at 100 kHz into the voltage input of the Autolab PGStat128N.

Reference is made to FIG. 10. Bode |Z| plots collected from a vancomycin-detecting EAB sensor immersed in whole bovine blood at 37° C. and challenged with increasing concentrations of vancomycin. The modulus of the impedance does not change significantly at any frequency as a function of target concentration, despite the observed phase shifts.

### Example 7.3 Filter Correction

The following experiment was performed to accurately measure impedance spectra by correcting for systematic artefacts in the experimental setup.

Reference is made to FIG. 11. Phase (blue) and modulus (green) Bode plots of a 10 kΩ resistor. Panel (A) shows the spectrum without filter corrections. |Z| is the expected 10 kΩ from 1 Hz to 1 kHz, but the phase is nonzero at high frequencies. Reference is made to panel (B) After correction, phase is zero across the frequency spectrum.

The low-pass current filter applied by the potentiostat may affect the measured impedance spectra and should be corrected for. To do this, the impedance spectrum of a 10 kΩ resistor (FIG. 8, panel A) was measured. While a constant impedance of 10 kΩ and a constant phase of 0° was expected for this resistor, a phase shift that reaches 7° at 1 kHz was measured. This is caused by the tail of the low-pass filter, which cannot be directly controlled on the potentiostat used in this study, but which is affected by the choice of current range. Subsequently, impedance spectra were corrected using this reference spectrum as shown in Equations S2 and S3:

$$\vert Z \vert ( ω ) = \frac{{\vert Z \vert}_{measured} ( ω )}{{\vert Z \vert}_{ref} ( ω )} \qquad ( S2 )$$

$$ϕ ( ω ) = {ϕ}_{measured} ( ω ) - {ϕ}_{ref} ( ω ) \qquad ( S3 )$$

Re-recording the impedance spectrum of the same 10 kΩ resistor and applying this correction procedure yielded the expected 10 kΩ |Z|, 0° phase across the entire spectrum (FIG. 8, panel B)

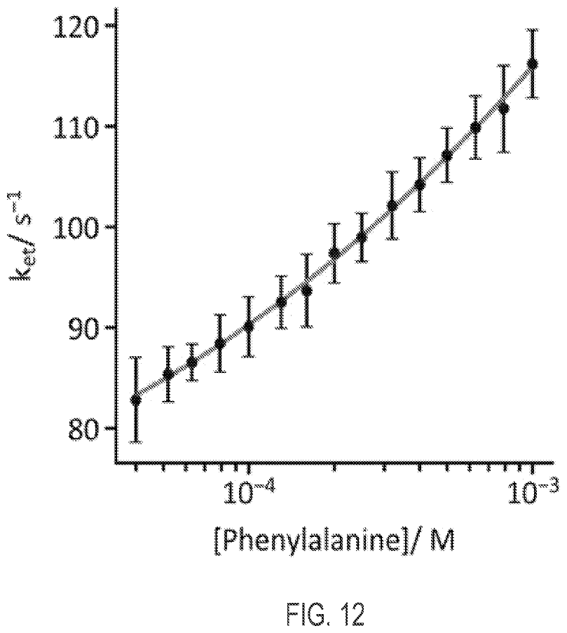

Reference is made to FIG. 12. The binding curve of a phenylalanine-detecting EAB sensor interrogated using EIS can be used to determine phenylalanine concentration from kₑₜ. To record this curve, the sensor was immersed in whole, freshly-collected rat's blood at 37° C. The endogenous concentration of phenylalanine in the blood (52 μM) was determined e using a fluorescent assay kit. Aliquots of phenylalanine dissolved in PBS-BSA were then added to the blood to increase the phenylalanine concentration. Values of kₑₜ (black points, error bars represent the standard deviation across four independently fabricated and tested sensors) fall on a Langmuir isotherm,

$${k}_{et} = {k}_{et , 0} + ( {k}_{e t , ma x} - {k}_{et , 0} ) \frac{{[ Phe ]}^{n}}{{K}_{D}^{n} + {[ Phe ]}^{n}} \qquad ( S4 )$$

where k_{et,0} is the value of kₑₜ in the absence of phenylalanine, k_{et,max} is the value of kₑₜ at saturating phenylalanine, K_{D} is the dissociation constant, and n is the Hill coefficient. Fitting to this equation yielded k_{et,0}=62.9 s⁻¹, k_{et,max}=225.8 s⁻¹, K_{D}=6.89 mM, and n=0.38.

### Example 8: Use of FFT-EIS to Interrogate In Vivo EAB Sensors to Monitor Plasma Lactate Concentrations

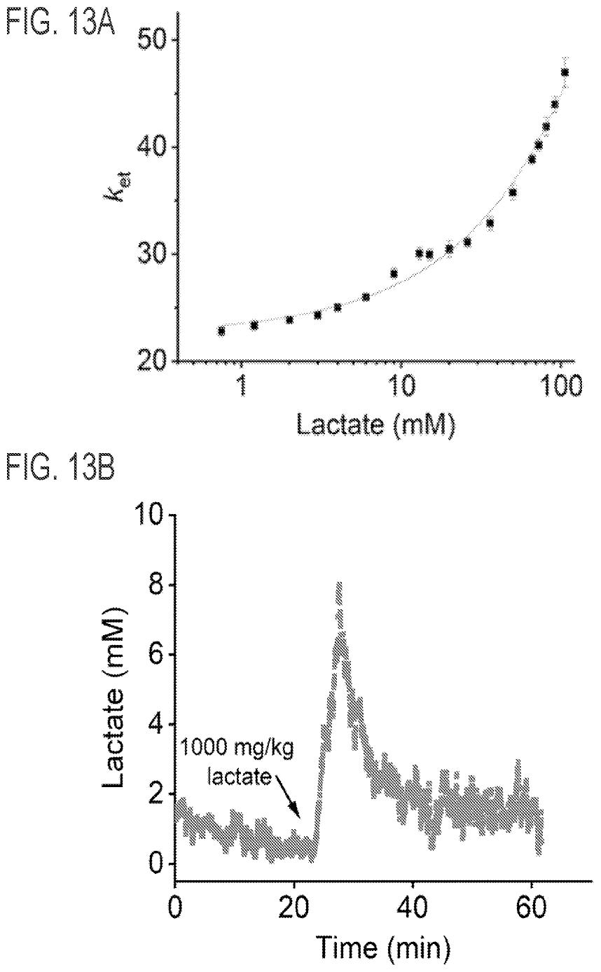

FFT-EIS can be used to interrogate in vivo EAB sensors to monitor plasma lactate concentrations in a live rat in real time (FIGS. 13A and 13B). Lactate is an important clinical biomarker for sepsis and hypoxia during anesthesia, and lactate levels are useful to monitor sport performances. FFT-EIS is used to characterize the lactate-detecting EAB sensor in undiluted blood held at about 37° C. by stepwise changing the lactate level and measuring the corresponding kₑₜ value (FIG. 13A). The electron transfer rate (kₑₜ) changes as function of the lactate level and shows a Langmuir binding isotherm. Lactate concentrations can be determined in real time in a living rat by measuring changes in kₑₜ when employing the lactate detecting EAB sensor (FIG. 13B). Stable concentrations were measured prior to intravenously infusing about 1000 mg/kg sodium lactate. Plasma lactate levels then rose before they gradually returned to the prior baseline as the lactate was degraded.

In vitro EAB sensors were fabricated by soldering a 4.5 cm gold wire to an electrochemical connector for connection to the potentiostat and insulated using 3.6 cm of heat shrinkable polyolefin tubing. The uncovered gold wire was cut to 6 mm.

To fabricate the electrodes of intravenous sensors, gold (0.2 μm diameter×10 cm in length; 99.9% purity), platinum (0.125 μm diameter×10 cm in length; 99.95% purity), and silver (0.125 μm diameter×10 cm in length; 99.99% purity) wires were cut and insulated with polytetrafluoroethylene heat-shrink (PTFE). The wires were bundled with physical gaps separating each wire to prevent shorting. The insulation was then trimmed to produce an exposed length of 3 mm (gold), 5 mm (platinum), and 1 cm (silver). To convert the silver wire to a reference electrode, the silver wire was submerged in 7.5% sodium hypochlorite (commercial bleach) overnight to form a stable silver chloride film. Finally, the electrodes were rinsed in Milli-Q water to remove any residual bleach.

To clean the gold, the wire was immersed first in a 0.5 M NaOH solution and subjected to electrochemical cleaning using a potential window from −1.0 V to −2.0 V (potentials versus Ag/AgCl) at a scan rate of about 1 V/s for 1000 cycles using a CH1040 C potentiostat in a three-electrode setup using a platinum counter electrode and a Ag/AgCl reference electrode. Next, the microscopic roughness of the gold wire was increased by placing the wire in a 0.5 M H₂SO₄ solution and pulsed from 0.0 V to 2.2 V using a pulse width of 0.02 s, which was repeated for about 32,000 cycles. The degree of surface roughening was verified by determination of the electrodes surface area in a 0.5 M H₂SO₄ solution using a potential window from 0 V to 1.8 V at a scan rate of 1 V/s for 10 cycles.

The lactate aptamer is modified with a six-carbon thiol and the redox reporter methylene blue. The aptamer was reduced for 1 h in a solution containing about 11.26 μM aptamer and 8.87 mM TCEP and subsequently diluted till 500 nM in a PBS buffer plus 2 mM MgCl₂. The intravenous sensors were fed through 20-gauge catheters and used the in vitro sensors as is. To form the lactate-detecting EAB sensor, the aptamer was deposited on the gold electrode by immersing the electrode for 1 h in a 500 nM reduced lactate aptamer solution followed by overnight immersion in 10 mM 6-mercaptohexanol to form a self-assembled monolayer. The finished sensor was washed with Milli-Q prior to use. Before use in vivo, the catheters were filled with 1×PBS.

The in vitro sensors were used for the calibration of the lactate-detecting EAB sensor in bovine blood with FFT-EIS. 2.5 mg/mL NaF was added to the blood to inhibit anaerobic glycolysis and stored the blood at about 37° C. for 2 hours prior to the calibration. The lactate detecting EAB sensors were immersed in the blood for about 45 min to obtain a stable baseline. FFT-EIS was performed on the Autolab PGStat128N. The endogenous lactate concentration was determined in blood prior to the start of the calibration with a lactate blood test kit. The lactate concentration in the blood sample was increased till 106 mM and stepwise lowered by diluting the lactate level in blood using blood without extra lactate and PBS plus 25 mg/mL BSA.

The in vivo experiments were performed using adult male Sprague-Dawley rats (4-5 months old, 300-500 g). These were pair-housed in a temperature and humidity-controlled vivarium on a 12-h light-dark cycle and provided ad libitum access to food and water. Rats were induced under 4% isoflurane gas in a Plexiglas anesthesia chamber. Anesthesia was maintained with 2-3% isoflurane gas/oxygen administered via a nose cone for the experiment's duration. A pulse oximeter was used to measure heart rate and SpO₂ during the experiment. The rat was shaved and the skin above the jugular vein was disinfected with 70% ethanol and betadine. A small incision was made to isolate both jugular veins. A small incision in the jugular vein was made using spring-loaded microscissors. A silastic catheter (composed of a bent steel cannula and silastic tubing) was inserted into the left jugular vein for infusions. The EAB sensor was inserted into the right jugular vein for in-vein lactate monitoring and stabilized with sterile 6-0 silk sutures. Following this insertion, 30 units of heparin were infused through the indwelling infusion line to prevent clotting at the surface of the electrode. Before lactate infusion, approximately 45 minutes were waited to establish a stable baseline. For lactate dosing, a 3 M stock of sodium lactate in 1× phosphate buffered saline was infused through the silastic catheter connected to a motorized syringe pump.

### Example 9: Discussion of Results Arising from the Experimental Work Described in Examples 2 Through 8

The experimental work detailed supra establishes FFT-EIS as a rapid and reliable interrogation method for EAB sensors, both in vitro and in vivo applications. Specifically, this work has demonstrated the ability of FFT-EIS to measure the electron transfer rate associated with EAB sensors and to use this rate to determine the concentration of target analytes with a time resolution of just 1.8 s. Because this approach uses kₑₜ as a means of monitoring target concentration (rather than absolute current), it is independent of both sensor-to-sensor fabrication variation and the drift arising due to fouling in biological fluids, rendering the technique suitable for performing calibration-free in vivo measurements. In support of this, vancomycin- and phenylalanine-detecting EAB sensors were demonstrated to successfully monitor plasma concentrations of these targets in the veins of live animals, with time resolution of better than 2 second and without requiring the calibration of each, individual sensor. When combined with the modularity of aptamers, the benefits associated with impedimetric interrogation of EAB sensors could improve understanding of pharmacokinetics, metabolism, disease progression, and neurochemistry, and play an important role in the future of personalized medicine.

In the present systems, any of the devices or servers may comprise network interface means configured to interface with other components of the system. The network interface means typically routes data into and out of a system component.

The methods and apparatus described herein may be deployed in part or in whole through one or more processors that execute computer software, program codes, and/or instructions on a processor. The processor may be part of a server, client, network infrastructure, mobile computing platform, stationary computing platform, or other computing platform. A processor may be any kind of computational or processing device capable of executing program instructions, codes, binary instructions and the like. The processor may be or may include a signal processor, digital processor, embedded processor, microprocessor or any variant such as a coprocessor (math co-processor, graphic co-processor, communication co-processor and the like) and the like that may directly or indirectly facilitate execution of program code or program instructions stored thereon. In addition, the processor may enable execution of multiple programs, threads, and codes.

The threads may be executed simultaneously to enhance the performance of the processor and to facilitate simultaneous operations of the application. By way of implementation, methods, program codes, program instructions and the like described herein may be implemented in one or more thread. The thread may spawn other threads that may have assigned priorities associated with them; the processor may execute these threads based on priority or any other order based on instructions provided in the program code. The processor may include memory that stores methods, codes, instructions and programs as described herein and elsewhere.

Any processor or a mobile communication device or server may access a storage medium through an interface that may store methods, codes, and instructions as described herein and elsewhere. The storage medium associated with the processor for storing methods, programs, codes, program instructions or other type of instructions capable of being executed by the computing or processing device may include but may not be limited to one or more of a CD-ROM, DVD, memory, hard disk, flash drive, RAM, ROM, cache and the like.

A processor may include one or more cores that may enhance speed and performance of a multiprocessor. In some embodiments, the processor may be a dual core processor, quad core processors, other chip-level multiprocessor and the like that combine two or more independent cores (called a die).

The methods and systems described herein may be deployed in part or in whole through one or more hardware components that execute software on a server, client, firewall, gateway, hub, router, or other such computer and/or networking hardware. The software program may be associated with a server that may include a file server, print server, domain server, internet server, intranet server and other variants such as secondary server, host server, distributed server and the like. The server may include one or more of memories, processors, computer readable media, storage media, ports (physical and virtual), communication devices, and interfaces capable of accessing other servers, clients, computers, and devices through a wired or a wireless medium, and the like. The methods, programs or codes as described herein and elsewhere may be executed by the server. In addition, other devices required for execution of methods as described in this application may be considered as a part of the infrastructure associated with the server.

The server may provide an interface to other devices including, without limitation, clients, other servers, printers, database servers, print servers, file servers, communication servers, distributed servers and the like. Additionally, this coupling and/or connection may facilitate remote execution of program across the network. The networking of some or all of these devices may facilitate parallel processing of a program or method at one or more location without deviating from the scope of the invention. In addition, any of the devices attached to the server through an interface may include at least one storage medium capable of storing methods, programs, code and/or instructions. A central repository may provide program instructions to be executed on different devices. In this implementation, the remote repository may act as a storage medium for program code, instructions, and programs.

The software program may be associated with a client that may include a file client, print client, domain client, internet client, intranet client and other variants such as secondary client, host client, distributed client and the like. The client may include one or more of memories, processors, computer readable media, storage media, ports (physical and virtual), communication devices, and interfaces capable of accessing other clients, servers, computers, and devices through a wired or a wireless medium, and the like. The methods, programs or codes as described herein and elsewhere may be executed by the client. In addition, other devices required for execution of methods as described in this application may be considered as a part of the infrastructure associated with the client.

The client may provide an interface to other devices including, without limitation, servers, other clients, printers, database servers, print servers, file servers, communication servers, distributed servers and the like. Additionally, this coupling and/or connection may facilitate remote execution of program across the network. The networking of some or all of these devices may facilitate parallel processing of a program or method at one or more location without deviating from the scope of the invention. In addition, any of the devices attached to the client through an interface may include at least one storage medium capable of storing methods, programs, applications, code and/or instructions. A central repository may provide program instructions to be executed on different devices. In this implementation, the remote repository may act as a storage medium for program code, instructions, and programs.

The methods and systems described herein may be deployed in part or in whole through network infrastructures. The network infrastructure may include elements such as computing devices, servers, routers, hubs, firewalls, clients, personal computers, communication devices, routing devices and other active and passive devices, modules and/or components as known in the art. The computing and/or non-computing device(s) associated with the network infrastructure may include, apart from other components, a storage medium such as flash memory, buffer, stack, RAM, ROM and the like. The processes, methods, program codes, instructions described herein and elsewhere may be executed by one or more of the network infrastructural elements.

The methods, program codes, calculations, algorithms, and instructions described herein may be implemented on a cellular network having multiple cells. The cellular network may either be frequency division multiple access (FDMA) network or code division multiple access (CDMA) network. The cellular network may include mobile devices, cell sites, base stations, repeaters, antennas, towers, and the like. The cell network may be a GSM, GPRS, 3G, 4G, EVDO, mesh, or other networks types.

The methods, programs codes, calculations, algorithms and instructions described herein may be implemented on or through mobile devices. The mobile devices may include navigation devices, cell phones, mobile phones, mobile personal digital assistants, laptops, palmtops, netbooks, pagers, electronic books readers, music players and the like. These devices may include, apart from other components, a storage medium such as a flash memory, buffer, RAM, ROM and one or more computing devices. The computing devices associated with mobile devices may be enabled to execute program codes, methods, and instructions stored thereon.

Alternatively, the mobile devices may be configured to execute instructions in collaboration with other devices. The mobile devices may communicate with base stations interfaced with servers and configured to execute program codes. The mobile devices may communicate on a peer-to-peer network, mesh network, or other communications network. The program code may be stored on the storage medium associated with the server and executed by a computing device embedded within the server. The base station may include a computing device and a storage medium. The storage device may store program codes and instructions executed by the computing devices associated with the base station.

The computer software, program codes, and/or instructions may be stored and/or accessed on computer readable media that may include: computer components, devices, and recording media that retain digital data used for computing for some interval of time; semiconductor storage known as random access memory (RAM); mass storage typically for more permanent storage, such as optical discs, forms of magnetic storage like hard disks, tapes, drums, cards and other types; processor registers, cache memory, volatile memory, non-volatile memory; optical storage such as CD, DVD; removable media such as flash memory (e.g., USB sticks or keys), floppy disks, magnetic tape, paper tape, punch cards, standalone RAM disks. Zip drives, removable mass storage, off-line, and the like; other computer memory such as dynamic memory, static memory, read/write storage, mutable storage, read only, random access, sequential access, location addressable, file addressable, content addressable, network attached storage, storage area network, bar codes, magnetic ink, and the like.

The methods and systems described herein may transform physical and/or or intangible items from one state to another. The methods and systems described herein may also transform data representing physical and/or intangible items from one state to another.

The elements described and depicted herein, including in flow charts and block diagrams throughout the figures, imply logical boundaries between the elements. However, according to software or hardware engineering practices, the depicted elements and the functions thereof may be implemented on computers through computer executable media having a processor capable of executing program instructions stored thereon as a monolithic software structure, as standalone software modules, or as modules that employ external routines, code, services, and so forth, or any combination of these, and all such implementations may be within the scope of the present disclosure.

The methods and/or processes described above, and steps thereof, may be realized in hardware, software or any combination of hardware and software suitable for a particular application. The hardware may include a general-purpose computer and/or dedicated computing device or specific computing device or particular aspect or component of a specific computing device. The processes may be realized in one or more microprocessors, microcontrollers, embedded microcontrollers, programmable digital signal processors or other programmable device, along with internal and/or external memory. The processes may also, or instead, be embodied in an application specific integrated circuit, a programmable gate array, programmable array logic, or any other device or combination of devices that may be configured to process electronic signals. It will further be appreciated that one or more of the processes may be realized as a computer executable code capable of being executed on a computer readable medium.

The Application software may be created using a structured programming language such as C, an object oriented programming language such as C++, python or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and software, or any other machine capable of executing program instructions.

Thus, in one aspect, each method described above and combinations thereof may be embodied in computer executable code that, when executing on one or more computing devices, performs the steps thereof. In another aspect, the methods may be embodied in systems that perform the steps thereof, and may be distributed across devices in a number of ways, or all of the functionality may be integrated into a dedicated, standalone device or other hardware. In another aspect, the means for performing the steps associated with the processes described above may include any of the hardware and/or software described above. All such permutations and combinations are intended to fall within the scope of the present disclosure.

The invention may be embodied in program instruction set executable on one or more computers. Such instruction sets may include any one or more of the following instruction types:

Data handling and memory operations, which may include an instruction to set a register to a fixed constant value, or copy data from a memory location to a register, or vice-versa (a machine instruction is often called move, however the term is misleading), to store the contents of a register, result of a computation, or to retrieve stored data to perform a computation on it later, or to read and write data from hardware devices.

Arithmetic and logic operations, which may include an instruction to add, subtract, multiply, or divide the values of two registers, placing the result in a register, possibly setting one or more condition codes in a status register, to perform bitwise operations, e.g., taking the conjunction and disjunction of corresponding bits in a pair of registers, taking the negation of each bit in a register, or to compare two values in registers (for example, to see if one is less, or if they are equal).

Control flow operations, which may include an instruction to branch to another location in the program and execute instructions there, conditionally branch to another location if a certain condition holds, indirectly branch to another location, or call another block of code, while saving the location of the next instruction as a point to return to.

Coprocessor instructions, which may include an instruction to load/store data to and from a coprocessor, or exchanging with CPU registers, or perform coprocessor operations.

A processor of a computer of the present system may include “complex” instructions in their instruction set. A single “complex” instruction does something that may take many instructions on other computers. Such instructions are typified by instructions that take multiple steps, control multiple functional units, or otherwise appear on a larger scale than the bulk of simple instructions implemented by the given processor. Some examples of “complex” instructions include: saving many registers on the stack at once, moving large blocks of memory, complicated integer and floating-point arithmetic (sine, cosine, square root, etc.), SIMD instructions, a single instruction performing an operation on many values in parallel, performing an atomic test-and-set instruction or other read-modify-write atomic instruction, and instructions that perform ALU operations with an operand from memory rather than a register.

An instruction may be defined according to its parts. According to more traditional architectures, an instruction includes an opcode that specifies the operation to perform, such as add contents of memory to register- and zero or more operand specifiers, which may specify registers, memory locations, or literal data. The operand specifiers may have addressing modes determining their meaning or may be in fixed fields. In very long instruction word (VLIW) architectures, which include many microcode architectures, multiple simultaneous opcodes and operands are specified in a single instruction.

Some types of instruction sets do not have an opcode field (such as Transport Triggered Architectures (TTA) or the Forth virtual machine), only operand(s). Other unusual “0-operand” instruction sets lack any operand specifier fields, such as some stack machines including NOSC.

Conditional instructions often have a predicate field-several bits that encode the specific condition to cause the operation to be performed rather than not performed. For example, a conditional branch instruction will be executed, and the branch taken, if the condition is true, so that execution proceeds to a different part of the program, and not executed, and the branch not taken, if the condition is false, so that execution continues sequentially. Some instruction sets also have conditional moves, so that the move will be executed, and the data stored in the target location, if the condition is true, and not executed, and the target location not modified, if the condition is false. Similarly, IBM z/Architecture has a conditional store. A few instruction sets include a predicate field in every instruction; this is called branch predication.

The instructions constituting a program are rarely specified using their internal, numeric form (machine code); they may be specified using an assembly language or, more typically, may be generated from programming languages by compilers.

Those skilled in the art will appreciate that the invention described herein is susceptible to further variations and modifications other than those specifically described. It is understood that the invention comprises all such variations and modifications which fall within the spirit and scope of the present invention.

Accordingly, the spirit and scope of the present invention is not to be limited by the foregoing examples, but is to be understood in the broadest sense allowable by law.

What is claimed is:

1. A method for determining an amount of an analyte in a sample comprising:
interrogating a working electrode of an electrochemical sensor by applying a voltage perturbation thereto, the voltage perturbation being a sum of two or more sinusoidal waveforms, each of the two or more sinusoidal waveforms being of a different frequency;
measuring voltage and/or current values across the working electrode and a counter electrode at each of the different frequencies simultaneously;
applying an integral transform method to the measured voltage and/or current values to generate an impedance spectrum by determining impedance at the frequencies of the two or more sinusoidal waveforms; and
using the impedance spectrum to determine the amount of analyte in the sample.

2. The method of claim 1, wherein the integral transform method is selected from a Fourier transform method, a fast Fourier transform method, a Laplace transform method, a Mellin transform method, a Hartley transform method, and a Chirplet transform method.

3. The method of claim 1 or claim 2, wherein the two or more sinusoidal waveforms are 3, 4, 5, 6, 7, 8, 9, 10, 11, 12, 13, 14, 15, 16, 17, 18, 19, 20 or more sinusoidal waveforms.

4. The method of any one of claims 1 to 3, wherein the frequencies of the two or more sinusoidal waveforms define a frequency range that includes frequencies informative of electron transfer kinetics between a redox reporter of the electrochemical sensor and a surface of the working electrode.

5. The method of any one of claims 1 to 4, wherein the frequencies of the two or more sinusoidal waveforms define a frequency range that excludes frequencies not informative of electron transfer kinetics between a redox reporter of the electrochemical sensor and a surface of the working electrode.

6. The method of any one of claims 1 to 5, wherein the frequencies of the two or more sinusoidal waveforms define a frequency range that excludes frequencies that result in a drift error in the determined analyte concentration.

7. The method of any one of claims 1 to 6, wherein the frequencies of the two or more sinusoidal waveforms are each less than 2000 Hz, 1900 Hz, 1800 Hz, 1700 Hz, 1600 Hz, 1500 Hz, 1400 Hz, 1300 Hz, 1200 Hz, 1100 Hz, 1000 Hz, 900 Hz, 800 Hz, 700 Hz, 600 Hz, 500, 400 Hz, 300 Hz, 200 Hz, or 100 Hz.

8. The method of any one of claims 1 to 7, wherein the frequencies of the two or more sinusoidal waveforms are each between 1 Hz and 2000 Hz, or are each between 1 Hz and 1000 Hz, or are each between 10 Hz and 100 Hz.

9. The method of any one of claims 1 to 8, wherein the frequencies of the two or more sinusoidal waveforms include a first frequency informative of electron transfer kinetics between a redox reporter of the electrochemical sensor and a surface of the working electrode, a second frequency that is higher than the first frequency, and a third frequency that is lower than the first frequency.

10. The method of any one of claims 1 to 9, wherein the frequencies of the two or more sinusoidal waveforms include a lower frequency and one or more frequencies higher than the lower frequency, and each of the one or more frequencies higher than the lower frequency is a multiple of the lower frequency.

11. The method of claim 10, wherein the lower frequency is the lowest of the frequencies of the two or more sinusoidal waveforms.

12. The method of any one of claims 1 to 11, wherein the step of using the impedance spectrum to determine the amount of analyte comprises comparing an impedance spectrum or part thereof resulting from a test sample, with an impedance spectrum or part thereof resulting from a control sample containing no analyte.

13. The method of claim 12, wherein the impedance spectrum resulting from a control sample and the impedance spectrum resulting from a test sample are both arranged as frequency versus phase.

14. The method of claim 12 or claim 13, wherein the impedance spectrum resulting from a control sample and the impedance spectrum resulting from a test sample each comprise a feature at a first frequency and a second frequency respectively, and wherein the amount of analyte is determined by reference to the difference between the first frequency and the second frequency.

15. The method of claim 14, wherein the feature is a peak or a maximum, a trough or a minimum, an upwardly slanting portion of the spectrum, or a downwardly slanting portion of the spectrum.

16. The method of any one of claims 1 to 15, wherein the step of using the impedance spectrum to determine the amount of analyte comprises using the spectrum to determine electron transfer kinetics between a redox reporter of the electrochemical sensor and a surface of the working electrode.

17. The method of claim 16, wherein the electron kinetics is an electron transfer rate between the redox reporter of the electrochemical sensor and the working electrode surface.

18. The method of any one of claims 1 to 17, capable of repeatedly determining a concentration of an analyte at an interval of less than 1 min, 50 seconds, 40 seconds, 30 seconds, 20 seconds, 10 seconds, 9 seconds, 8 seconds, 7 seconds, 6 seconds, 5 seconds, 4 seconds, 3 seconds, 2 seconds, or 1 second.

19. The method of any one of claims 1 to 18, comprising repeatedly determining a concentration of an analyte at an interval of less than 1 min, 50 seconds, 40 seconds, 30 seconds, 20 seconds, 10 seconds, 9 seconds, 8 seconds, 7 seconds, 6 seconds, 5 seconds, 4 seconds, 3 seconds, 2 seconds, or 1 second.

20. The method of any one of claims 1 to 19, comprising repeatedly determining a concentration of an analyte for at least 1 hour, 2 hours, 3 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 30 hours, 36 hours, 42 hours, or 48 hours.

21. The method of any one of claims 1 to 20 that does not require any frequency sweeping step across a frequency range to determine the amount of analyte in a sample.

22. The method of any one of claims 1 to 21 that does not require any calibration step to determine the amount of analyte in a sample.

23. The method of any one of claims 1 to 22 that does not require any drift adjustment step to determine the amount of analyte in a sample.

24. The method of any one of claims 1 to 23, wherein the sample is a bodily fluid within or about the body of a subject.

25. The method of claim 24, wherein the bodily fluid is selected from: interstitial fluid (ISF), blood, saliva, a lacrimal secretion, a lactational secretion, a nasal secretion, a tracheal secretion, a bronchial secretion, an alveolar secretion, a gastric secretion, a gastric content, a glandular secretion, a vaginal secretion, a uterine secretion, a prostate secretion, semen, urine, sweat, cerebrospinal fluid, a glomerular filtrate, an hepatic secretion, bile, and an intraocular fluid.

26. The method of any one of claims 1 to 25, wherein the working electrode is a wire, a needle, or a microneedle.

27. The method of any one of claims 1 to 26, wherein the electrochemical sensor comprises a recognition element configured to specifically recognize a target analyte.

28. The method of claim 27, wherein the recognition element is associated with a redox reporter.

29. The method of claim 27 or claim 28, wherein the recognition element and/or the redox reporter undergo a change in the presence of the target analyte, the change altering a rate of electron transfer between the redox reporter and a surface of the working electrode.

30. The method of claim 29, wherein the change in the recognition element is a conformational change.

31. The method of claim 30, wherein the conformational change in the recognition element alters a distance between the redox reporter and a surface of the working electrode.

32. The method of claim 30 or claim 31, wherein the conformational change in the recognition element alters a reorganization energy of the redox reporter.

33. The method of any one of claims 28 to 32 wherein a rate at which the redox reporter approaches a surface of the working electrode is altered in the presence of the target analyte.

34. The method of any one of claims 28 to 33, wherein a fraction of time that the redox reporter is proximal to a surface of the working electrode rather than distal is altered in the presence of the target analyte.

35. The method of claim 33 or claim 34, wherein the alteration in rate or the fraction of time is associated with a target analyte associated alteration in a steric bulk parameter, a biomolecular rigidity parameter, an electrostatic parameter, or a hydrodynamic radius of the redox reporter.

36. The method of any one of claims 29 to 35, wherein the change in the recognition elements and/or the redox reporter is a dissociation of the recognition element from the target analyte.

37. The method of any one of claims 30 to 36, wherein the conformational change alters a rate of electron transfer between a redox reporter associated with the recognition element and a surface of the working electrode.

38. The method of any one of claims 27 to 37, wherein the recognition element is associated with a surface of the working electrode, and the redox reporter is associated with the recognition element and the conformational change in the recognition element alters a distance between the redox reporter and the surface of the working electrode which in turn alters a rate of electron transfer between the redox reporter and the surface of the working electrode.

39. The method of any one of claims 27 to 38, wherein the recognition element is a biological polymer.

40. The method of claim 39, wherein the biological polymer is a nucleic acid.

41. The method of claim 39 or claim 40, wherein the biological polymer is an aptamer.

42. The method of any one of claims 1 to 41, wherein the electrochemical sensor is configured as a wearable device.

43. Apparatus for detecting an amount of an analyte in a test sample, the apparatus comprising:
an electrochemical sensor having a working electrode and a counter electrode;
a power source configured to apply a voltage perturbation to the working electrode, the voltage perturbation being a sum of two or more sinusoidal waveforms, each of the two or more sinusoidal waveforms being of a different frequency;
a voltage and/or a current measuring circuit connected across the working electrode and the counter electrode; and
a processor configured to integrally transform a measured voltage and/or current to generate an impedance spectrum, and to use the impedance spectrum to determine the amount of analyte in the test sample.

44. The apparatus of claim 43, wherein the processor has access to program instructions configured to execute the method of any one of claims 1 to 42.

45. The apparatus of claim 43 or claim 44, wherein the electrochemical sensor comprises a redox reporter, and the processor has access to program instructions configured to execute the method of claim 4 or claim 5.

46. The apparatus of any one of claims 43 to 45, wherein the working electrode is a wire, a needle or a microneedle.

47. The apparatus of any one of claims 43 to 46, wherein the electrochemical sensor comprises a recognition element configured to specifically recognize a target analyte.

48. The apparatus of claim 47, wherein the recognition element is associated with a redox reporter.

49. The apparatus of claim 47 or claim 48, wherein the recognition element and/or the redox reporter undergo a change in the presence of the target analyte, the change altering a rate of electron transfer between the redox reporter and a surface of the working electrode.

50. The apparatus of claim 49, wherein the change in the recognition element is a conformational change.

51. The apparatus of claim 50, wherein the conformational change in the recognition element alters a distance between the redox reporter and a surface of the working electrode or a coupling constant describing electron transfer through the recognition element.

52. The apparatus of claim 50 or claim 51, wherein the conformational change in the recognition element alters a reorganization energy of the redox reporter.

53. The apparatus of any one of claims 48 to 52, wherein a rate at which or fraction of time during which the redox reporter approaches a surface of the working electrode is altered in the presence of the target analyte.

54. The apparatus of any one of claims 48 to 53, wherein a fraction of time that the redox reporter is proximal to a surface of the working electrode rather than distal is altered in the presence of the target analyte.

55. The apparatus of any one of claims 49 to 54, wherein the alteration in rate or the fraction of time is associated with a target analyte associated alteration in a steric bulk parameter, a biomolecular rigidity parameter, an electrostatic parameter, or a hydrodynamic radius of the redox reporter.

56. The apparatus of any one of claims 49 to 55, wherein the change in the recognition elements and/or the redox reporter is a dissociation of the recognition element from the target analyte.

57. The apparatus of any one of claims 50 to 56, wherein the conformational change in the recognition element alters a rate of electron transfer between a redox reporter associated with the recognition element and a surface of the working electrode.

58. The apparatus of any one of claims 47 to 57, wherein the recognition element is associated with a surface of the working electrode, and the redox reporter is associated with the recognition element and the conformational change in the recognition element alters a distance between the redox reporter and the surface of the working electrode which in turn alters a rate of electron transfer between the redox reporter and the surface of the working electrode.

59. The apparatus of any one of claims 47 to 58, wherein the recognition element is a biological or biomimetic polymer.

60. The apparatus of claim 59, wherein the biological polymer is a nucleic acid.

61. The apparatus of claim 59 or claim 60, wherein the biological polymer is an aptamer.

62. The apparatus of any one of claims 43 to 61, wherein the electrochemical sensor is configured as a wearable device.

63. Non-transitory computer-readable media comprising computer-executable program instructions configured to execute the method of any one of claims 1 to 42.

64. The apparatus of any one of claims 43 to 62, wherein the program instructions are provided by the non-transitory computer-readable media of claim 63.


SEQUENCE LISTING (2 sequences)

SEQ ID NO: 1 - DNA, 45 bp, synthetic construct
  source 1..45: mol_type = other DNA
cgagggtaccgcaatagtacttattgttcgcctattgtgggtcgg

SEQ ID NO: 2 - DNA, 38 bp, synthetic construct
  source 1..38: mol_type = other DNA
cgaccgcgtttcccaagaaagcaagtattggttggtcg